(19) United States
(12) Patent Application Publication
(10) Pub. No.: US 2025/0285755 A1
(43) Pub. Date: Sep. 11, 2025

(54) Cardiovascular Disease Classification and Management Using Artificial Intelligence

(71) Applicant: Board of Regents of the University of Nebraska, Lincoln, NE (US)

(72) Inventors: John R. Windle, Omaha, NE (US); Ketemwabi Yves Shamavu, Omaha, NE (US); Thomas A. Windle, Omaha, NE (US)

(21) Appl. No.: 18/600,688

(22) Filed: Mar. 9, 2024

Related U.S. Application Data
(60) Provisional application No. 63/489,730, filed on Mar. 10, 2023.

(51) Int. Cl. G16H 50/20

(52) U.S. Cl. CPC G16H 50/20

(57) ABSTRACT

Technology is described for identifying hypertension in a person. The method can include identifying a first group of medical features for a person at a first time point and a second group of medical features for the person at a second time point. An additional operation may be determining a time difference interval between the first time point and the second time point. The first group of medical features may be processed using an initial Bayesian belief network. An initial hypertension classification may also be received from the initial Bayesian belief network. In a further operation, the second group of medical features may be processed with an additional Bayesian belief network, while using the initial hypertension classification and the time difference interval as inputs. A joint hypertension classification may be obtained from the additional Bayesian belief network.

### RELATED APPLICATION

This application claims priority to U.S. Provisional Application No. 63/489,730 filed Mar. 10, 2023, entitled “Cardiovascular Disease Classification Using Machine Learning” which is incorporated herein by reference.

### BACKGROUND

Cardiovascular disease affects nearly half of American adults. Hypertension affects more that 100 million Americans, coronary artery disease affects over 20 million, heart failure 6.5 million and atrial fibrillation affects over 3 million Americans. As an example, hypertension is one of the world's leading factors in cardiovascular disease. Forty-seven percent or close to one in two Americans aged 18 and older are affected. It predicts approximately a thousand deaths per day. Based on recent statistics from the Centers for Disease Control and Prevention, one in three patients with hypertension does not know they are hypertensive. Despite robust guidelines and performance measures, these diseases are underdiagnosed and undertreated. For example, data from Nebraska Medicine deidentified records indicates nearly 70% of outpatient (ambulatory visits) would be classified as hypertension using the ACC/AHA Hypertension Guidelines, however, only 19% of the patients have a clinical diagnosis of hypertension.

Seventy-five percent of hypertensive patients have uncontrolled hypertension—meaning that they are not treated to target. While there is extensive literature on hypertension diagnosis and management, there is an apparent gap in understanding and acknowledging that a person is hypertensive. Moreover, blood pressure in a patient is not constant and can cover all the four hypertension stages delineated by the 2017 American College of Cardiology/American Heart Association at varying times in a day, week or month.

### BRIEF DESCRIPTION OF THE DRAWINGS

FIG. 1 illustrates example features that may be used in a Bayesian network for the present technology.

FIG. 2 is a block diagram illustrating an example of cleaning operations on data.

FIG. 3 illustrates an example of an equation for computing the ASCVD risk.

FIG. 4 illustrates an example of data cleaning steps for the time series.

FIG. 5 illustrates an example of a grouped time series hierarchy diagram.

FIG. 6 illustrates an example of a count-based time series hierarchy diagram.

FIG. 7 illustrates an example of a first recurrent neural network architecture.

FIG. 8 illustrates an example of a second recurrent neural network architecture.

FIG. 9 illustrates an example of a long short-term memory cell arrangement.

FIG. 10 illustrates an example of hierarchical diagrams of four count-based time series spinoffs.

FIGS. 11 and 12 depict examples of the performance of the two recurrent neural network (RNN) variants.

FIG. 13 illustrates an example of a simplified directed acyclic graph.

FIG. 14 illustrates an example of a directed acyclic graph with a set of vertices and edges.

FIG. 15 illustrates an equation for a combinatorial approach to joint probability distribution.

FIG. 16 illustrates an example equation for Bayes' rule.

FIG. 17 illustrates an example equation for marginalizing over the vertices not involved in the query.

FIG. 18 illustrates an example equation for the chain rule.

FIG. 19 illustrates a Bayesian belief network directed acyclic graph.

FIG. 20 depicts an example of vertices connected through a number T of time steps.

FIG. 21 depicts a confusion matrix where both the two-step dynamic Bayesian network and recurrent neural network models achieved perfect accuracy on the test set.

FIG. 22 depicts the distribution of labels for the two-step spinoff.

FIG. 23 is a flowchart illustrating an example of a method for identifying a cardiovascular condition.

FIG. 24 is a flowchart illustrating an example of a method for identifying a cardiovascular condition using a time difference interval.

FIG. 25 is a flowchart illustrating an example configuration for a causal inference process.

FIG. 26 is a block diagram illustrating an example of flow of data and decisions for the hypertension use cases.

FIG. 27 illustrates that blood pressure recordings are dynamic and are based on the individual's situation and clinical condition.

FIG. 28 is a block diagram that provides an example illustration of a computing device that may be employed in the present technology.

### DETAILED DESCRIPTION

Reference will now be made to the examples illustrated in the drawings, and specific language will be used herein to describe the same. It will nevertheless be understood that no limitation of the scope of the technology is thereby intended. Alterations and further modifications of the features illustrated herein, and additional applications of the examples as illustrated herein, which would occur to one skilled in the relevant art and having possession of this disclosure, are to be considered within the scope of the description.

Some groups have explored and developed software-based tools to diagnose hypertension. Such tools are often made available on the Internet and serve patients or Internet users who are curious about their hypertension stage. Vendors of modern blood pressure cuffs also run hypertension-related analytics for Bluetooth-enabled cuffs that transmit data to the computation engine on an end-user's computer or smartphone device. Yet, such solutions are not useful when a physician or advanced practice provider (APP) is conducting a diagnosis or deciding on the best management plan. Moreover, these public solutions are not developed in light of the latest guidelines and performance measures.

The present technology can improve the accuracy of predicting the clinical phenotype for hypertension based on causal inferences. Causality depends on constructing a knowledge base and representation of causal effects. By building models based on clinical guidelines and validating the models through independent experts, models may be built that accurately predict the clinical phenotype. Causal inference in artificial intelligence may be an extension of clinical abductive reasoning. Causal interference as described by Judea Pearl may inform three levels of causation: association, inference by doing and the counter factual argument.

A dynamic artificial intelligence model can be used to depict a person's blood pressure's variation over time. The model may employ an upstream machine learning model to classify blood pressure stages used by the downstream dynamic model. The upstream model predicted hypertension stage with nearly 100% fidelity. Four different variants of the final dynamic models, going from two to five steps were successfully trained and tested using two different approaches: Deep learning recurrent neural network and dynamic Bayesian belief network. The causal inference engine may be used to support not only the diagnosis of the cardiovascular condition (e.g., hypertension) but also support the management of treatments (such as antihypertensive medications) to get the patient treated to target.

This technology can improve the diagnosis, management and support for the treatment of cardiovascular conditions through use of a causal inference engine, deep learning recurrent neural networks and/or dynamic Bayesian belief network. This technology has at least two useful improvements over existing solutions. First, causal mechanisms support transparency and understanding of the process which is valuable for physician acceptance. Second, the technology supports continuous versus dichotomous definitions. Thus, two measurements at two settings in the hypertension range will not automatically label the patient as hypertensive. In addition, current guidelines and performance measures do not adequately capture the complexity of clinical care. They rely on dichotomous decision points rather than a more nuanced and granular view of multiple inputs including causal relationships and dynamic data.

This technology provides processes and systems for assisting in the process of developing clinically-oriented artificial intelligence models for hypertension diagnosis, as an example of chronic cardiovascular conditions. The use of dynamic artificial intelligence algorithms can model the problem given the fluctuating nature of blood pressure over time. The data used may be obtained from outpatient clinic visits. Upstream machine learning can classify the visits into blood pressure stages used for the downstream dynamic models. The upstream models' predicted stages revealed a statistically significant (p=0.0001) difference with hypertension status. In one example, four different variants of the final dynamic models, going from two to five time steps, were trained and used two different approaches: deep learning recurrent neural network (RNN) and dynamic Bayesian belief network.

This technology can provide a hypertension diagnosis tool using transparent, interpretable artificial intelligence (AI) trained on data curated using recent hypertension guidelines and clinical performance measures. A challenging aspect of clinical electronic record systems is the automation of the problem list curation, i.e., ensuring the changing patients' concerns and diagnoses are accurately reflected without duplicates or missing entries and in a timely fashion with less human resource (manpower) involved. The hypertension classification system can act as adaptive decision support providing predetermined insights to nudge physicians and advanced practice providers (APPs) to the right management plan decision.

### Data Preprocessing and Feature Extraction

The data sets and features used will be described in more detail. A description will also be provided about how machine learning can be employed to automatically classify data set records into blood pressure stages.

A data-centric approach can improve hypertension diagnosis accuracy and be an adaptive clinical decision support tool. Another aspect of this approach can be ensuring that the output of AI (artificial intelligence) models can be well explained and interpreted by clinicians. Knowledge bases founded on robust data-centric clinical concepts that are understandable to medical clinicians and computer scientists may be useful in optimizing artificial intelligence models. Models that achieve high predictive accuracy often employ deep learning or ensemble learning techniques whose outputs are not as transparent to the end-users. Data-centric approaches hold the potential to bring transparency and explainability to these high-performing but commonly named ‘black box’ techniques owing to their lack of transparency. Moreover, regularly curating machine learning input data to derive high-quality data from the raw data captured in day-to-day real-world business settings is valuable to the final output. Curating data captured in a real-world clinical environment to transform the data into high-quality data can serve to train AI algorithms to improve the accuracy of hypertension diagnoses.

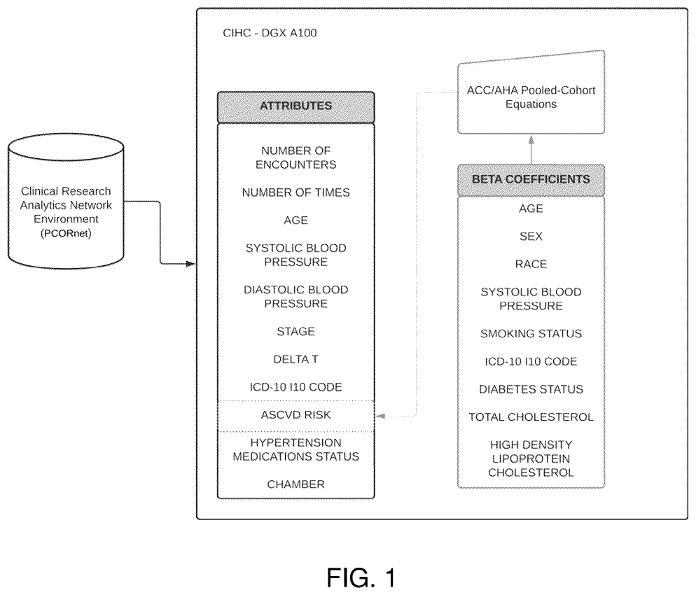

Good data for training machine learning models may include a curated set consisting of one or more of the following features: the number of encounters (or hospital visits), the number of times blood pressure was measured per encounter, age, systolic and diastolic blood pressures, hypertension stage, the difference in days between encounters (to account for the irregular time series), the documented ICD-10 hypertension status, the ASCVD (atherosclerotic cardiovascular disease) risk score, and/or hypertension medications status. In addition, the patient identification may be used to track the records and the corresponding output. And for time series data, the date and time of the last blood pressure recording during an encounter was tracked to order patient-specific encounters. FIG. 1 provides an illustration of examples of these attributes. The number of encounters may be used as a feature for accurate classification of hypertension stages but not in the dynamic models. Each encounter constitutes a discrete time step. Hence, it may not be necessary to retain the number of encounters as an additional feature.

### Exclusions

In addition to inclusions, it is important to document exclusions to get a more accurate numerator and denominator. A set of disease exclusions are considered for any disease with ICD-10 codes N17 (acute kidney failure), N18.5 (Stage 5 chronic kidney disease), N19 (unspecified kidney failure), Z94.0 (kidney transplant), or Z33.3 (pregnant state, gestational carrier). The date on which the diagnosis was established, the service date, and the measurement period may also be part of the attributes. Although home and ambulatory blood pressures can also be mapped, the data will typically consist of only clinic-based blood pressure determinations.

The data preparation process may start with known cleaning steps, i.e., removing null (or missing) and erroneous values. The number of encounters per patient, the number of times blood pressure was measured per encounter, and age may be derived from existing data columns. Further, all systolic blood pressure measurements greater than 300 millimeters of mercury may be considered outliers after consultation with cardiovascular domain experts. Similarly, for diastolic blood pressure, values greater than 150 millimeters of mercury may be removed.

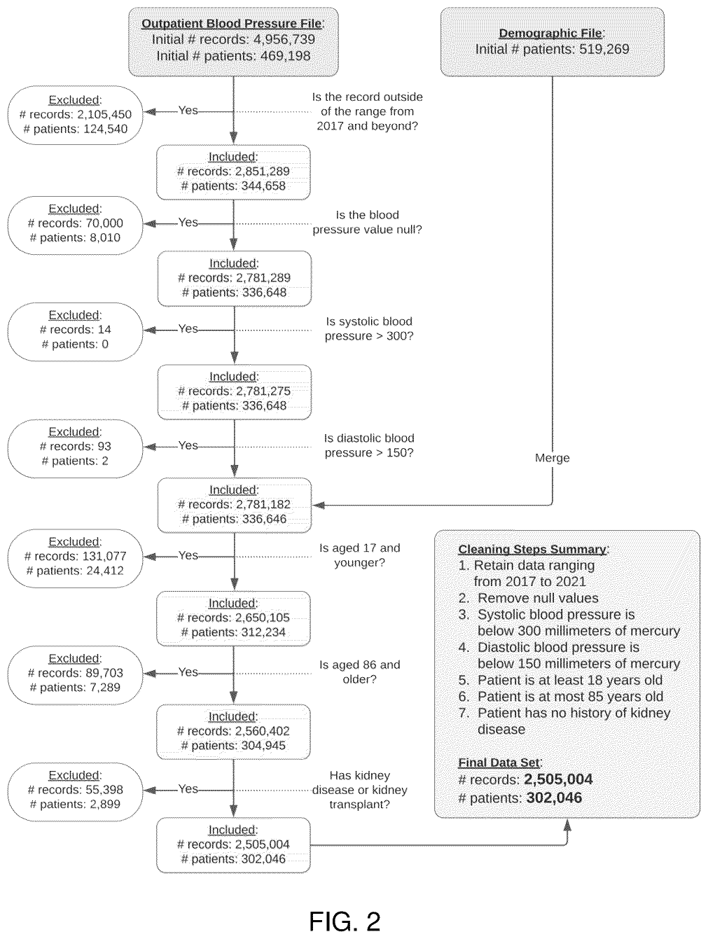

FIG. 2 illustrates an example of the cleaning steps. The number of blood pressure readings and patient records excluded are shown at each step. A summary of the final data set filtering conditions and the number of blood pressure readings and patient records are shaded in gray at the bottom of the figure.

### Derived Features

Age may be computed from the date of birth column in the demographics file. The 2017 ACC/AHA clinical practice guidelines suggest that hypertension affects individuals aged 18 and older. Moreover, when the clinical guidelines were published, the ACC/AHA was cognizant of the lack of randomized controlled trials for patients older than 85 years old. Therefore, the ACC/AHA recommended age group for a high accuracy prediction of hypertension was used, which goes from 18 to 85 years old. Patients with other serious health problems may also be excluded. For example, patients with an end-stage renal disease or a kidney transplant may be excluded.

The following question was asked for the number of encounters: how many encounters per patient are in the blood pressure records file? An aggregation by the patient can make it easier to compute the number of encounters per patient. Conversely, aggregating by the encounter column allowed derivation of the number of times by answering the question: how many times was blood pressure taken per encounter? It ensued that while most patients (more than 98%) in the data set were seen in the clinic between one and 80 times (with a median of 21 times), blood pressure was taken twice or less for about 95% of the encounters. Moreover, patients' ages ranged from 18 to 85 years old.

### Upstream Machine Learning

In the clinical research analytics environment data, the ACC/AHA blood pressure stages are not associated with the systolic and diastolic blood pressure records; the blood pressure stages are calculated based on the ACC/AHA Hypertension Clinical Guidelines. The guidelines provide clear-cut rules to generate the blood pressure stages based on the recorded measurements. This process can use machine learning to automatically classify blood pressure records into the different 2017 hypertension guideline stages. The techniques may include a Decision tree and/or Bayesian belief network classifications, which are transparent and interpretable probabilistic graphical models.

Simple rules inspired by the hypertension clinical practice guidelines may allow this process to label each corresponding systolic and diastolic blood pressure with the appropriate stage without requiring machine learning or sophisticated AI approaches. However, using machine learning has at least three advantages. It enables generalization of the classification automatically to new cases as the patients' blood pressure measurements are recorded by generating the corresponding stage without any extra steps from the attending physician or advanced practice provider. It may enforce an implicit adoption of the hypertension clinical practice guidelines. It may also consider other features beyond the blood pressure measurements by looking at the number of encounters and how often the measurements were recorded per encounter.

### Input Features

At least five features have been used in this classification. The five features used to train machine learning models in classifying blood pressures into stages may include the patient's age, systolic and diastolic blood pressure, the number of encounters, and the number of times the measurements were taken per encounter. The demographics file may include the date of birth from which age was computed. Age can be the difference between the date of the last clinic visit and the patient's birth date.

Thus, the features can include the number of encounters, the number of times measurements were taken per encounter, the patient's age, and the systolic and diastolic blood pressure measurements. Several features have displayed slight skewness and a slight difference in their respective scales. Hence, the features may be scaled using a scaling function since standardization is useful for features on different scales. For example, the scaling may subtract the average from each value of a given feature and divide the result by the standard deviation leading to unit variance. However, there was no performance difference between the scaled and non-scaled features.

Given their categorical nature, the generated labels were transformed into one-hot vectors. However, a single ordinal scale numeric value per category has proven to perform just as well as a one-hot coded category since the difference between the categories is meaningful for this task. Indeed, normal (or 0) is less of a concern than stage 1 (or 2). The performance of the classifiers was assessed using the accuracy, recall, and precision scores on an independent test set. The precision score is the fraction of true labels out of the total instances classified as (but not necessarily) belonging to a given class (true positives over true positives plus false positives). For example, if there were 100 total instances in a data set, among which 80 had the label “normal” and 20 had the label “other.” A classifier assigned 70 instances to the class “normal,” but ten instances among the 70 are false positives. The precision score would be (70 minus 10) divided by 70 or 0.85. The recall score measures how many positive classifications there are over the entire instances with the true label in the data set (true positives over true positives plus false negatives). Drawing from the previous example, the recall score would be how many instances are classified as “normal” out of all the “normal” instances in the data set, or (70 minus 10) divided by 100, i.e., 0.6. The accuracy score measures how many true positive and true negative instances over the total number of instances in the data set. In the previous example, the accuracy score would be ((70 minus 10) plus 20) divided by 100, or 0.8.

### Decision Tree Classifier

Decision tree classifiers are an attractive machine learning approach that can provide high efficiency in space and time performance. As long as the number of classes is not so large, a decision tree classifier can reach highly accurate results in less time compared to single-stage classifiers, which do not employ a tree-like classification behavior of splitting the decision across several branches. Moreover, the multi-stage decision pruning of a decision tree classifier can break down a complex decision-making problem into more manageable problems by distributing the processing over different levels of the tree. Each level of the tree can reach its local prediction by discriminating classes against the provided features. And thus, the stage-based classification can contribute to the prediction of the overarching classification problem.

Another key reason to establish a baseline accuracy using a decision tree classifier stems from its quality of being straight-forward to interpret as one can trace back the decision from the root node.

### Bayesian Belief Network Classifier

A Bayesian belief network classifier contrasts with a naive Bayes classifier, which employs a simple Bayesian network structure where every child vertex is dependent on one common parent vertex and is assumed to be independent of every other child vertex. However, a Bayesian belief network classifier encompasses child vertices that may depend on more than one parent vertex, and it does not have a strong independence assumption among all child vertices with respect to their parent vertices. The Bayesian belief network classifier algorithm learns conditional probability tables of a given network structure and employs the maximum likelihood estimation to classify instances across specified labels.

Bayesian networks have two main characteristics. The first is a directed acyclic graph, also referred to as its structure, which shows a graphical representation of the features in the model. The edges usually represent the relationships between the features, attributes, vertices, or domain variables, and the vertices represent the attributes themselves. The second characteristic is that each link in the directed acyclic graph is quantifiable by a conditional probability distribution between the corresponding feature or vertex and its parent. Bayesian analysis has at least two advantages over other forms of analysis: 1) Bayesian analysis does not presume a Gaussian distribution of values and 2) prior values may inform future predictions. This framework may be individual to the patient.

In one example, aGrUM/pyAgrum, a Python library may be used for building probabilistic graphical models and algorithms, to conduct parameter learning based on the Bayesian network directed acyclic graph structure. Specifically, a maximum likelihood estimator may be used to learn the parameters according to the Bayesian network structure with five vertices corresponding to the five features we considered to infer blood pressure stages. A lazy propagation technique may be used to derive inferences from the learned parameters while evaluating the model's performance on the test set.

### Feature Generation

Apart from the derived and upstream machine learning model inferred features, as described above, it can be useful to generate the ASCVD (atherosclerotic cardiovascular disease) risk score as an additional feature that can influence hypertension status. This section describes the step taken to engineer the ASCVD risk score.

We already considered medication status as one of the features since blood pressure-lowering medication can influence blood pressure trends over time and lead to false-negative hypertension status. Similarly, the ASCVD risk score is useful to detect hypertension early on. This risk is a ten-year hazard for developing cardiovascular disease, and it determines when a patient should start therapy. Hence, there is a direct dependency relation between ASCVD risk and whether a patient is on cardiovascular medications including hypertension.

Considering the ASCVD thus allows a proactive approach with an AI model that can detect hypertension in patients with an increased risk score without necessarily having had repeatedly high blood pressure. The ASCVD itself is a preventive metric. It is far out in time and if a patient is identified as being at a high risk of ASCVD without aggravating factors, then the nonpharmacological therapy would include activities that help with hypertension prevention as well.

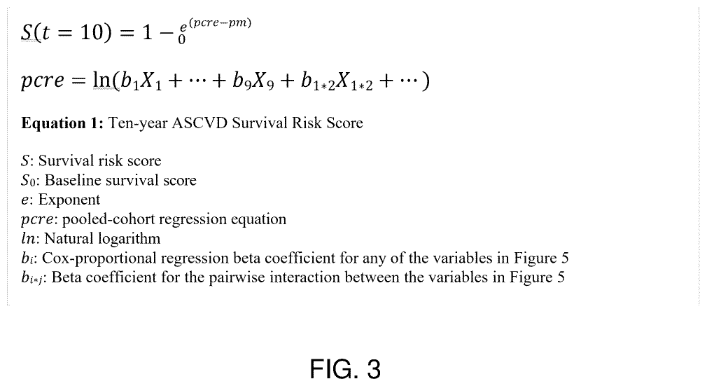

The ASCVD risk is computed by the formula in Equation 1 in FIG. 3. This equation was developed and published by the ACC/AHA in 2013. The ACC/AHA employed cohorts from the National Health and Nutrition Examination Survey. From these cohorts, they constructed pools based on participants race and sex because cardiovascular disease affects women and men and people from different races differently. Using the Cox-proportional regression, the ACC/AHA developed pooled-cohort equations and published the related beta coefficients. These published coefficients may be used along with the baseline survival and the pool-specific mean to generate the ASCVD risk feature.

### Labels

Accurate hypertension status hinges on a number of components, including: high blood pressure (which include stage one and two hypertension), the presence or absence of ICD-10 I10 code for essential hypertension on the problem list, and whether or not a patient is on medications. Table 1 illustrates the different combinations of these components which result in eight chambers or labels.

TABLE 1
Eight-Chamber Labels for Granular Hypertension Representation

| | High | | | | |
|---|---|---|---|---|---|
| | Blood | ICD-10 | Hypertension | | |
| Chamber | Pressure | I10 Code | Medication | Ideal | Modifiers |
| 1 | 0 | 0 | 0 | Normotensive | Up to 10% masked |
| | | | | | hypertension |
| 2 | 0 | 1 | 0 | Controlled | False positive |
| | | | | hypertension with non- | diagnosis |
| | | | | pharmacologic | |
| 3 | 0 | 1 | 1 | Controlled | |
| | | | | hypertension | |
| 4 | 0 | 0 | 1 | Medications prescribed | Failure to document |
| | | | | for non-hypertensive | hypertension as a |
| | | | | diagnosis | problem |
| 5 | 1 | 0 | 1 | Hypertensive but | |
| | | | | undocumented | |
| | | | | hypertension | |
| 6 | 1 | 1 | 1 | Uncontrolled | |
| | | | | hypertension | |
| 7 | 1 | 1 | 0 | Untreated hypertension | Discrepancy between |
| | | | | | clinic and home blood |
| | | | | | pressure |
| | | | | | measurements |
| 8 | 1 | 0 | 0 | Undiagnosed/Untreated | Up to 30% white coat |
| | | | | hypertension | hypertension or |
| | | | | | inappropriate |
| | | | | | technique |

The presence of a documented diagnosis on the problem list is considered in the dynamic model because it allows the model to play the assistive role or act as a decision support tool. Indeed, to get closer to a truly adaptive decision support AI model, the decision the clinician has made as to whether they believe a patient is hypertensive can be considered and the AI model can support the clinician with a prediction regarding the accuracy of their decision and important modifiers supported by evidence from published and peer-reviewed research. This not only gets us closer to adaptive decision support as the chamber will change from visit to visit based on the fluctuations in hypertension or blood pressure stage when the documented ICD-10 code remains static, but also it fulfills an aspect of the problem-knowledge coupler. Essentially, using the documented diagnosis couples (or links) the physician's decision to published literature.

Hence, the AI model may not just improve the diagnosis of hypertension, but it also assists with management by linking the predictions to modifiers and alleviates the cognitive load of physicians and APPs by providing information that enhances recall. For instance, chamber one might mean the patient is not just normotensive but there is up to 10% of chance they have masked hypertension. This prompt allows the physician to request adequate follow ups to rule out edge cases. In this scenario, it may be decided to employ home or ambulatory blood pressure monitoring devices which are not prone to masking the patient's hypertension stage, i.e., a low blood pressure reading in clinic when the patient might have a high reading away from the clinic.

The chamber categories are ordered such as those with less clinical concern appear first. It is a convenient ordering to help when experimenting with machine learning models since some algorithms may assume that the order of the categories is significant. Otherwise, we also experimented with one-hot vectors in lieu of the categories. In fact, data can have four different measurement scales. It could be in either nominal, ordinal, interval, or ratio scale. Analytical techniques on categories such as the chambers in Table 1 naturally treat such categories as having an ordinal measurement scale, i.e., chamber two is of greater significance than chamber one. Hence, the current ordering in Table 1 considers such an assumption. A patient in chamber three would be a less severe case than a patient in chambers four, five, etc., where later chambers are considered more severe. However, a categorical variable can be encoded as a one-hot vector or as a dummy variable for greater scalability.

The encoding is especially needed when one knows that there is no ordinal relationship between the different categories. For instance, for the eight chambers, a one-hot encoding would represent each chamber as a one-dimensional vector with eight values. Chamber one would be [1, 0, 0, 0, 0, 0, 0, 0], chamber two would be [0, 1, 0, 0, 0, 0, 0, 0], etc., flipping the zero to a one for the corresponding numerical category. The eighth zero would become one for the eighth chamber when maintaining all previous entries to zero. Similarly, a dummy variable works the same way as a one-hot encoding, except it can help save memory by having eight minus one (8−1) one-hot vectors whereby none of the eight zeros are flipped to one in the last category.

### Benchmark Model

Deep learning techniques may be used to establish benchmark accuracy. This section presents a summary of data preprocessing and related figure. It then covers recurrent neural network (RNN) concepts and the long short-term memory layer. It also details how the training and validation of the model may be performed.

### Time Series Data

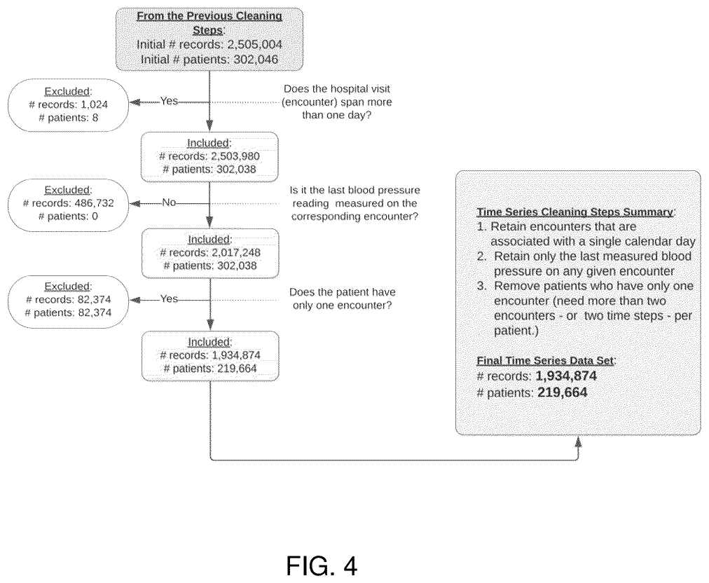

A cleaned data set can be employed in the training of the dynamic models. FIG. 4 illustrates data cleaning steps for the time series. A blood pressure record may be documented at the point of care and bears the encounter date and the time the documentation took place. The records can then be sorted ascendingly based on the encounter date and the documentation time. The ensuing time series is termed a discrete time series because it is encounter-based. The time points at which blood pressure measurements are recorded constitute a discrete set. A discrete time series contrasts with a continuous time series. For the latter, a collection of time points encompasses continuous observations over specified time intervals.

For instance, observations recorded via electrocardiography constitute a continuous time series. Modern and experimental wearable blood pressure recording devices also allow continuous blood pressure recording. The output of wearable, sensor-based blood pressure monitors is thus a continuous time series. Moreover, a continuous time series is the default output of the gold standard means for blood pressure recording. It is achieved through arterial cannulation using intra-arterial catheters.

The rise and fall of blood pressure over time is patient-specific. The records may be sorted based on the encounter date and measured time result in interwoven time points observations for the patients. Hence, the patient-specific blood pressure trend is obscured and begs for disaggregation. Thus, a time series data set made of encounter-based records for making hypertension predictions over time is discrete and multilevel. Each level comprises patient-related time series records. The discrete nature is due to the fact that all blood pressures are recorded in an outpatient clinic using blood pressure cuffs, a combination that does not permit a continuous time series. Hence, the data set is a discrete multilevel time series in this case.

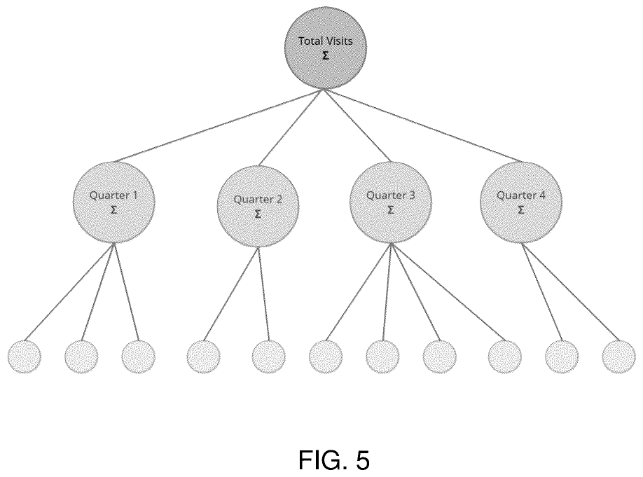

FIG. 5 illustrates a grouped time series hierarchy diagram. The top level is the aggregate number of hospital visits. It is divided by quarter (in the middle level), which is further divided into a more granular aggregation at the bottom level. However, the most common time series data sets covered in the statistical and machine learning literature include single-level, hierarchical, and grouped time series. A single-level time series consists of a data set with records sorted ascendingly from an earlier to a later time point without any grouping or separation level. A hierarchical time series is akin to a grouped time series. FIG. 5 illustrates a grouped discrete time series. The depiction is evocative of a hierarchy. But there is no hierarchical order inherent to the data set. Indeed, a grouped time series is structured like a hierarchical time series without a unique hierarchical structure because the order in which the individual series can be grouped is not unique.

For instance, in a task to predict the number of hospital visits on a given day, the data could consist of a retrospective time series collected a few years back. If we simply order the data from the minimum date to the maximum date, we could conduct time series analysis to forecast hospital visits on a single future date. However, we could group the data set records by nesting records under the corresponding quarter to create a two-level grouped time series similar to the one depicted in FIG. 5. The middle level would encompass the quarterly hospital visit totals. And the top level would be the aggregated total hospital visits from all the entries in the data set. Such a grouped time series implies more than one way to group the data in a nested fashion. Indeed, for the example in question, we could also group weekly, monthly, bi-annually, etc. However, a hierarchical time series means the data set records can only be nested in one unique way. In fact, we could group hospital visits by specialty instead of time periods. Since there is only one instance for each possible medical specialty, there would be only one unique hierarchy in the time series.

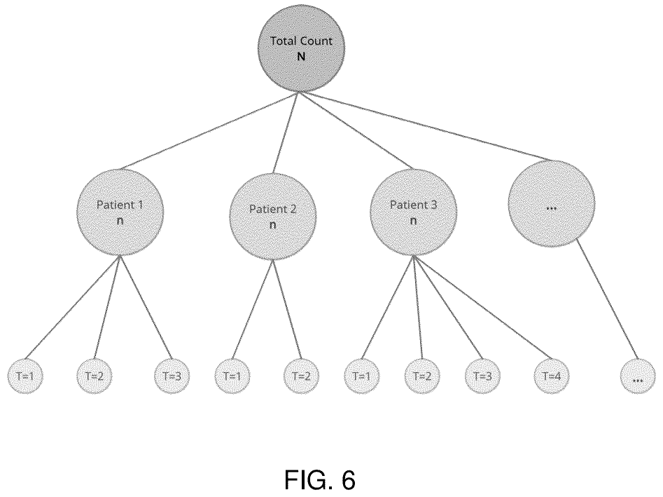

Nevertheless, the prediction of hypertension status based on the eight-chamber labeling does not lend itself to any plausible aggregation. Instead, each patient carries their own set of time series blood pressure records. Hence, the records were grouped by patient for the resulting sets of time series to constitute sequences with predictive power of hypertension status for the corresponding patient. The time series structure for this project is depicted in FIG. 6. At the top level, we have the ungrouped, disaggregated full set of records.

At the next level, there are patient-based groupings. The number of records per patient corresponds to the patient-specific total number of encounters. The bottom level represents ascending time points (patient-related time series) wherein each encounter date and time for each blood pressure reading per patient is a discrete time step. The last level shows that the time steps culminate into hypertension status prediction for the corresponding patient.

FIG. 6 illustrates an example of a count-based time series hierarchy diagram. The total count of time steps is divided by patient in the middle level, which, in turn, is divided by individual time steps corresponding to encounters (hospital visits) for a given patient. Patients may have an unequal number of encounters. Each discrete time step corresponds to an encounter. The value N represents: the total count of time steps, and the value n represents: patient-specific count.

An AI model is as good as the quality of the data used to train it. But insofar as the goodness of the data is already established, the next obvious gauge of the excellence of an AI model's performance is how it compares to a previously established performance. Traditionally, challenging AI problems are published or made otherwise publicly available. Researchers formulate solutions wherein the highest performance attainable is made publicly known as the benchmark performance for a corresponding problem. This allows AI scientists looking at improving on the performance to construct a better architecture.

In contrast to the benchmark performance, the baseline performance is a basic standard or attainable performance with a minimal model architecture. The model used to achieve the baseline performance is sometimes referred to as a zero rate (or ZeroR) model. A ZeroR model ignores other predictors and is merely tuned toward the majority class, i.e., achieving high accuracy for the label with the highest observation count or with the highest frequency in the data set. Other basic models based on different heuristics (other than the majority class) can provide a baseline accuracy. A random rate model is an example of such alternatives to the ZeroR model. It is also known as weighted guessing and employs knowledge from a prior class distribution to make a random class assignment. A basic model based on a simple heuristic is especially needed when tackling a problem with no established benchmark.

For this technology, benchmark accuracy was achieved using a simple recurrent neural network (RNN) with one hidden layer. A recurrent neural network is a class of neural networks that are suitable for time series analysis and forecasting. A hidden layer is any layer in the middle of a neural network (not the input nor the output layer). It consists of a stack of artificial neurons or threshold logic units that compute a weighted sum of inputs then applies an activation function.

Since transparent AI models are believed to be well suited to critical applications, the desire is to achieve a high-performing probabilistic model whose structure is determined by a panel of experts in cardiovascular medicine. For that reason, it is valuable to investigate the best possible accuracy attainable with non-transparent models such as deep learning models. Beyond establishing a baseline, a benchmark performance against which to compare the manually modeled probabilistic graphical algorithm may be obtained. Recurrent neural network (RNN) and deep learning approaches do not generally provide the ability to determine the structure of the model in the likeness of a directed acyclic graph for Bayesian network or probabilistic graphical approaches. However, while the Bayesian network allows the application of domain expertise to derive a suitable model for the said domain, a wrong assumption about the relationship between the vertices (or features) can lead to a sub-optimal model. Hence, the need to establish a baseline with an approach that, though undemanding of careful manual wiring of the influence between features, is prone to achieving high accuracy.

Deep learning is a type of machine learning that achieves great power and flexibility by learning to represent the world as a nested hierarchy of concepts and representations, with each concept defined in relation to simpler concepts, and more abstract representations computed in terms of less abstract ones. Baseline accuracy may be established by harnessing the greater power of deep learning to derive the most accurate predictions on the development data set and use this performance to gauge how well a manually modeled probabilistic approach compares. The comparison informs us on the exactness of the structure of the probabilistic graphical model derived from subject matter experts. For instance, if the recurrent neural network model outperforms the probabilistic graphical model, it would prompt further brainstorming with subject matter experts to question and manually restructure the probabilistic graphical model.

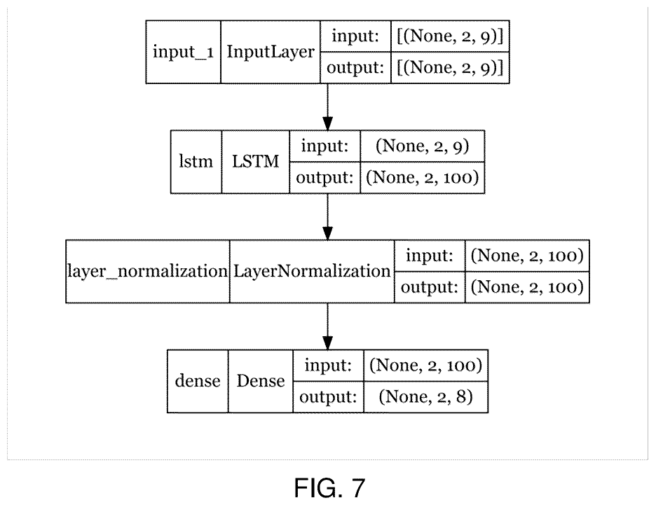

At least two recurrent neural network (RNN) variants may be used. The architecture of one variant is depicted in FIG. 7. It includes an input layer followed by a long short-term memory, then a layer normalization, and finally the output layer. The long short-term memory layer carries out all the recursive computations (processing forward and backward feeds of the input signal). The layer normalization normalizes across the feature dimensions. Layer normalization may transform the inputs at each time step into a Gaussian distribution, i.e., a mean activation of zero and a mean standard deviation of one for the activations at a given time step independently from a single input instance. Moreover, the normalization layer can estimate statistics mentioned above across the training set instances without requiring an exponentially moving average. It is recommended to apply the normalization layer after the hidden states. Indeed, its application before the hidden long short-term memory layer may yield a poor performance. The output layer comprises eight units and can produce a probability distribution over the eight chambers representing the likelihood of hypertension given the inputs at a specified time step.

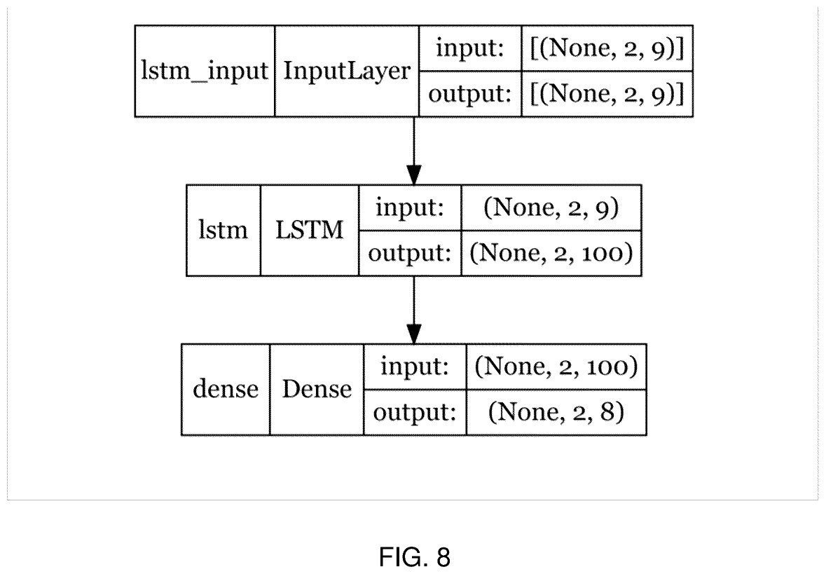

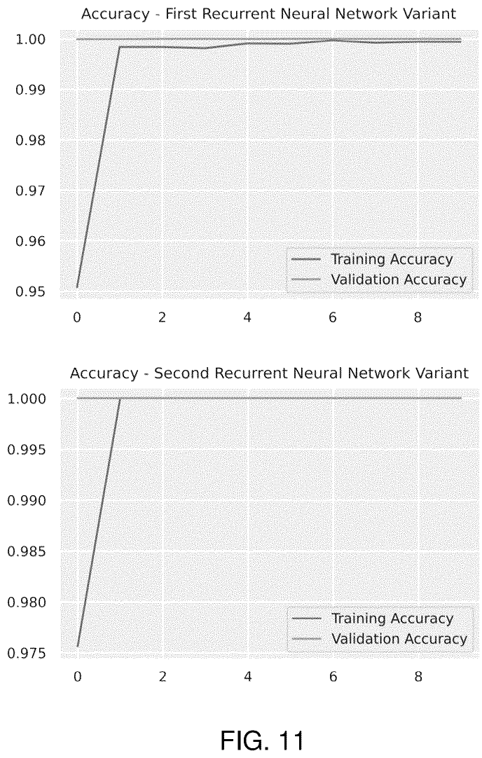

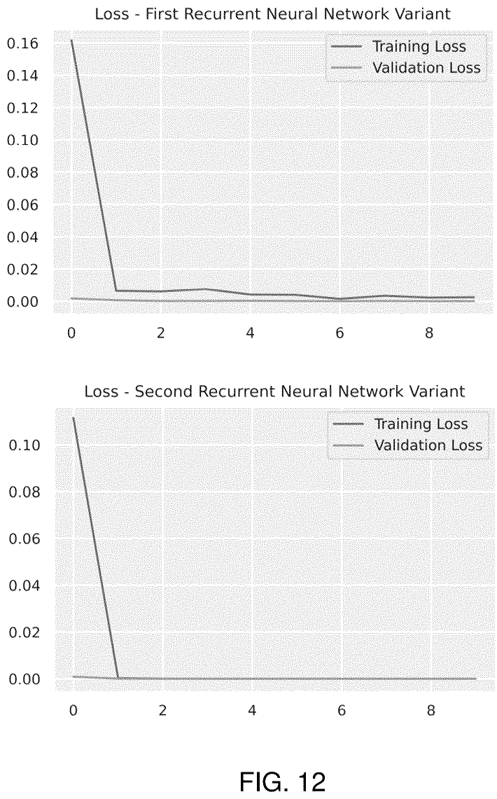

FIG. 8 illustrates another recurrent neural network variant. This variant lacks the implementation of the layer normalization. The model includes the long short-term memory layer followed by the output layer. However, the inputs coming through this variant can be normalized beforehand. The normalization function transforms the inputs to be within the range of zero and one. Normalizing the input features yielded a much more stable model compared to implementing a layer normalization, as illustrated in FIGS. 11 and 12. This further supports previous research showing that recurrent neural networks using long short-term memory are very sensitive to the scale of the input features.

### The Long Short-Term Memory Layer

Prior to the development of the long short-term memory layer, recurrent neural networks employed techniques such as backpropagation through time or real-time recurrent learning. Backpropagation through time is a variant of the backpropagation algorithm. It roughly works by initializing a set of weights to some random or arbitrary values. Then, the output is computed along with the error vector or the residuals between the actual output and the predicted output. The derivatives of the residual errors with respect to the weights are thus computed. If the errors increase by increasing the weights, then the weights are decreased. However, if the errors decrease by increasing the weights, then the weights are increased. For a standard backpropagation algorithm, the derivatives are calculated for the previous and current weights in a single forward pass through the network. However, in backpropagation through time, the derivatives are also based on previous time steps.

Hence, to maintain the network's feedforward nature, the recurrent network is unfolded over time. The parameters are shared across time steps, thus allowing gradient descent, i.e., the gradual measure of the change in all the weights with regard to the change in the errors at the current time step to reach an optimum specified by a given cost function. This leads to the blowing up of error signals that are flowing back to previous time steps to compute the required derivations. It is especially true when dealing with long sequences spanning a large number of time steps. Moreover, the entire sequence must be processed before backpropagating the errors and updating the weights, leading to a memory explosion.

Real-time recurrent learning calculates, during the forward step, the derivatives of errors and outputs with respect to the current and previous weights as the sequence is being processed by the recurrent neural network. This approach is dubbed “real-time” since the derivatives at the current time step are computed simultaneously with the outputs at the current time step from derivatives at the previous time step and the inputs at the current time step. Hence, with recurrent real-time learning, the model's parameters are calculated online without the requirement of storing the entire sequence, and as such, the recurrent neural network needs not be unfolded. However, this requires the learning rate to stay very low, thus dramatically increasing the running time. The memory requirement for storing the step-wise updates may grow exponentially with time. Moreover, the gradients are prone to vanish when bridging time steps from a very long sequence.

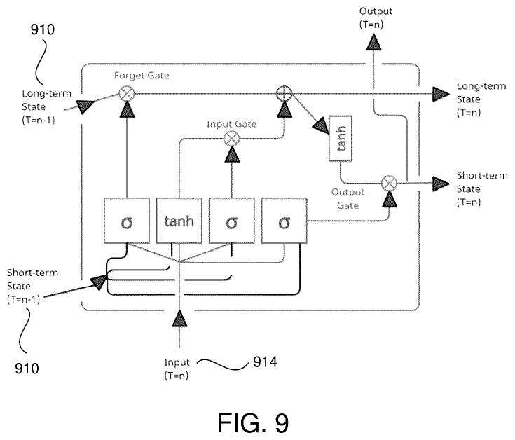

The long short-term memory helps solve these issues with backpropagation through time (whether truncated or not) and real-time recurrent learning approaches. It maintains a long-term state without losing the short-term time lag capabilities. The long short-term memory also employs a gradient-based algorithm for parameter updates; however, it enforces a constant non-vanishing and non-exploding error flow by implementing special internal states within a long short-term memory cell. The long short-term memory cell arrangement is depicted in FIG. 9. It takes in three inputs: a long-term state from the previous time step 910, a short-term state from the previous time step 912, and the input features of the current time step 914. In FIG. 9, the + represents addition, σ represents a Logistic function that rescales data between 0 and 1, and tanh represents a function that rescales data between −1 and 1. The four successive functions toward the bottom of the cell are fully connected with the input from the current step (T=n) 914 and the short-term state from the previous step (T=n−1) 910.

The short-term state from the previous time step 910 and the input features at the current time step 914 go through transformations carried out by four subsequent fully connected layers: a primary layer and three layers acting as gate controllers. One of the gate controller's processes inputs from the previous short-term state along with the feature inputs at the current time step. Its outputs dictate what part of the long-term state from the previous time step should be dropped. The second fully connected layer acting as a gate controller also takes in inputs from the previous short-term state along with the feature inputs at the current time step. It outputs a vector that is combined with the outputs of the primary layer. This combination constitutes an input gate that controls what parts of the input features and the previous short-term state should be added to the previous long-term state. After this addition, the long-term state is sent straight out as the current time step's long-term state. However, a copy of it is made to constitute a new short-term state. The last fully connected layer takes the same inputs as the other two gate controllers, and its outputs form the output gate. The latter controls what parts of the copy of the long-term state should be dropped. The result is thus sent out as the current time step's short-term state. The output gate also dictates which parts of the long-term state form the prediction at the current time step. A state is also referred to as memory.

Since recurrent neural networks are a special case of artificial neural networks that also employ deep learning to learn patterns across a temporal sequence, understanding some concepts about deep learning can be helpful. Deep learning help mimic the workings of human neural networks. Indeed, deep learning employs gradient descent to learn parameters, activation functions to carry out different input transformations, and an objective function for optimal parameter learning.

Activation functions are used because the performance of a neural network depends on the type of activation function used during training, given the number and type of layers used. In the absence of an activation function, a neural network acts the same as a linear regression algorithm by modeling the conditional mean of the output as a linear function of the input.

Nonetheless, a linear activation function might be sufficient for very simple problems. Although, for complex problems, even when several hidden layers are stacked together, a linear combination of their outputs would result in another linear function. Hence, non-linear activation functions are necessary to tackle problems where changes in the output are not proportional (or linear) to the changes in the input.

### Gradient Descent and Backpropagation

Backpropagation applies techniques from the stochastic descent learning method to parameterized neural networks. In other words, backpropagation uses gradient descent to compute gradients of the objective function with respect to the network's parameters to minimize or maximize the objective function while propagating the error backwards through the network. Hence, stochastic gradient descent iteratively updates the network's parameters until the objective function reaches an optimum point.

### Objective Functions and Learning Rate

In deep learning, an objective function is any function used during training for gauging when the machine has reached the optimal prediction. It could be a cost function when the goal is to minimize it to achieve optimal prediction or a reward function when the goal is to maximize it for optimal learning. Furthermore, the objective function is called a cost function when it is an average loss over a set of instances. Otherwise, if the objective function concerns only a single instance, it is referred to as a loss function. Objective functions are fundamental optimization techniques not only for deep learning applications but also for other machine learning and mathematical modeling techniques. Indeed, it is possible to formulate any machine learning task as an optimization technique. For an AI model to learn from experience, the objective function is needed to ensure the learning is completely automatic without human intervention. In a sense, the objective function is designed to provide feedback to the model on how to improve the learning process for better performance.

In gradient descent, the objective function's shape plays a role in how efficient the learning process will be. A convex cost function, for instance, ensures that gradient descent reaches the global minimum with high certainty compared to a multimodal objective function. This also shows the necessity of a suitable learning rate. When the learning rate is high, gradient descent takes steps of disproportionate size resulting in an erratic learning process before the model converges. On the other hand, if the learning rate is low, gradient descent is slow to reach convergence. A model is said to converge when gradient descent reaches the global minimum. It is thus valuable to have an appropriately tuned learning rate value when training a deep learning model. In fact, there are objective functions that can help a model fine-tune the learning rate during training ensuring the value assigned is conducive to optimality. This optimization technique of hyper-parameter tuning during learning is called meta-learning or teaching the model to learn how to learn in order to achieve good performance.

### Training and Validating a Recurrent Neural Network

Supervised machine learning can be used in the training of the recurrent neural network. In simpler terms, the goal of supervised learning is to adapt the parameters of the recurrent neural network so that the predicted labels (Ŷ) come close to the provided labels or target output (Y). The adapted parameters are thus expected to achieve a performance similar to the one attained on the training set, given some new unseen patterns in a different set. For each time series, the labels were provided in categorical format.

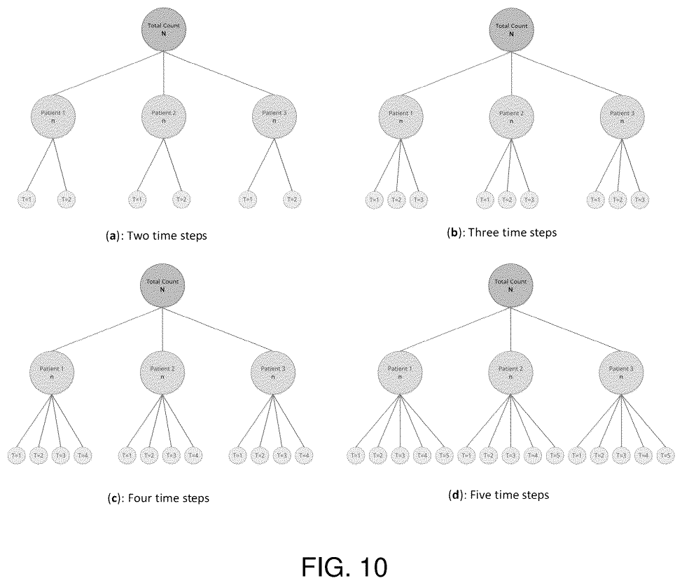

Such types of labels are designated as sparse labeling because the classes are mutually exclusive, i.e., a sampled instance can belong to exactly one class. Each recurrent neural network variant was in turn trained on four data set spinoffs (i.e., the four by-products of the initial data set represented in FIG. 10). The time series structure varied from one data set spinoff to the other by respectively truncating the third level to two time steps, three time steps, four time steps, or five time steps. FIG. 10 illustrates the four spinoffs with different number of time steps in the third level. Each spinoff resulted in a different model, given the variations in the data set.

One optimization technique employed for recurrent neural network variants is a sparse cross-entropy cost function. The cross-entropy is a type of objective function that a model minimizes to achieve optimum performance. Entropy is a notion derived from information theory. It conveys a measure of information or bits involved in the representation of a randomly drawn event or observation from a probability distribution. It can thus help identify whether a distribution is skewed or biased or whether it is fair or balanced. In the former case, there would be less entropy as a majority event will be very likely to occur in a skewed distribution. However, there would be high entropy in the latter case of balanced distribution as each event would have the same probability of being observed. Hence, a surprising information implies that the event is not widely available and carries a high entropy. Consequently, unsurprising information concerns events with low entropy.

Nevertheless, during a supervised training of a deep learning model, the goal is to minimize the cross-entropy function. Indeed, in supervised learning, labels are provided, in this case, zero or one. The probability of either zero or one in the original labels is unsurprising since we knew it before training the model. The model seeks to approximate a target probability of labels distributed identically to the original labels. Hence, we have an expected probability distribution from the original labels and a predicted probability distribution from the predicted labels. It follows that to demonstrate good performance, a model must minimize the cross-entropy cost function across the training batches so that the two distributions can become similar by the end of the training.

In one example, a learning rate of ten to the power of minus three was specified, which is the default learning rate for the gradient descent approach we employed. The Adam optimization algorithm, an extended version of stochastic gradient descent, was used. The Adam optimization algorithm employs first-order gradient descent to reach the global minimum during training based on stochastic objective functions. This algorithm is part of a set of algorithms introduced to improve the plain stochastic gradient descent of the 1960s. Indeed, due to an increase in the size of data sets brought about by the big data era, plain stochastic gradient descent proved to be tremendously slow when training a very large neural network.

FIGS. 11 and 12 depict the performance of the two recurrent neural network variants. The variant corresponding at the bottom of each figure demonstrates a low performance compared to its counterpart. The depicted performance is of the two step time series spinoff. The performance did not vary, if at all, between the different time series spinoffs depicted in FIG. 10.

### Probabilistic Modeling

Transparent AI models allow for interpretability as a direct consequence of their inherent design, i.e., without requiring any extra steps while designing the model. This contrasts with opaque AI models, which often provide accurate but unfathomable predictions by virtue of the complex connections in their hidden states facilitated by a series of transformations through activation functions.

Interpretability may be defined as: the degree to which a human can understand the cause of a decision. However, the approach to probabilistic graphical modeling employed in this technology, although it could model causality, is bounded to its direct conditional relationships modeling. Indeed, a directed acyclic graph in a Bayesian network readily depicts influence flow between adjacent vertices and mediation flow between two vertices with the same child vertex. However, it can be enhanced to represent an order of events whereby parents and child vertices are chronologically bound such that the edges depict the direction of time, which may imply causation since an effect could not occur prior to its cause.

Although, dynamic Bayesian networks carry a temporal aspect, interpretability, for the purpose of a straightforward probabilistic model, can also be defined as: the degree to which a human can consistently predict the model's result. Therefore, this definition renders the distinction between transparent and opaque AI models more crisply. The probabilistic modeling approach employed allows easy inspection of the predictions as long as one possesses basic knowledge on the interpretation of conditional probability tables. As a corollary, interpretability further implies that the interpreter has domain knowledge of the probabilistic approach employed. Also, different probabilistic graphical modeling approaches have differing degrees of interpretability depending on how much domain knowledge is needed to interpret the results.

In addition, transparent AI modeling begs for clarification between interpretability and explainability. As already established, interpretability has a spectrum from barely interpretable to greatly interpretable depending on the domain knowledge required for the specific probabilistic modeling approach. The more interpretable a model, the closer it gets to being explainable, and the lesser AI domain knowledge is required. While interpretability may apply to a batch of predicted outputs, explainability can better be assessed on individual predictions, especially when there is an added layer of transparency whereby not only is the output displayed to the end-user but also a user-friendly explanation of the significance of the output and the role each input feature played for the given output.

Data-centric health care is useful for unbiased and accurate application of analytical techniques because of the high-stakes nature of medicine. But it is evident that there resistance when it comes to adopting AI in actual clinical practice unless the ability to interpret and explain the predictions of the analytical techniques are provided. Transparent AI modeling is thus an ingredient in increasing the use of data-centric health care. The accuracy and explainability of transparent AI can incentivize continuous efforts to curate good data, which in turn fuels transparent AI, a relationship that can further actualize truly evidence-based health care.

Blackbox or non-transparent models can be rendered explainable and interpretable via additional layers of complexity with techniques such as local interpretable model-agnostic explanation and Shapley additive explanations to generate explanations, and feature importance and accumulated local effects, for generating interpretations. Nonetheless, such techniques increase the compute time and suffer from other drawbacks, unlike the native interpretability of probabilistic graphical models, which may be conducive to faster explanations.

### Concepts of Bayesian Networks

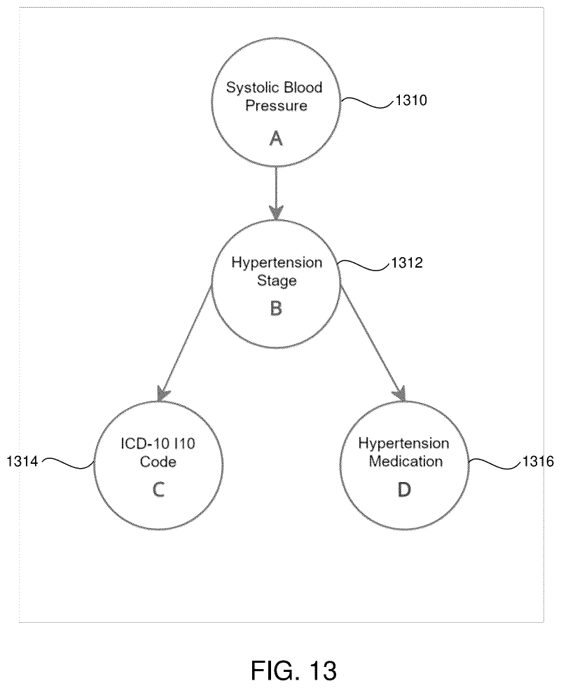

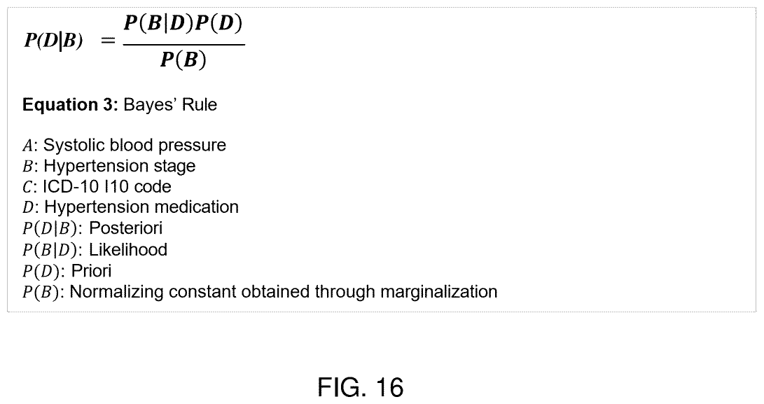

The probabilistic graphical modeling approach for this technology employs Bayesian networks. A Bayesian network consists of vertices, (also called) nodes or random variables, and edges or arrows. The vertices are random variables. Together, they model conditional probability relationships, edges denoting the relationships between vertices. The conditional probability relationships can be a direct application of Bayes' rule. Let's consider, for instance, two vertices: the patient's systolic blood pressure (A) 1310 and the patient's hypertension stage (B) 1312. As depicted in FIG. 13, the edge shows a relationship going from A to B, implying that A influences B. As per Equation 3 in FIG. 16, the Bayes' rule can be formulated as follows: the probability of B given A (known as the posterior probability) equals the probability of B (also known as the prior probability) times the probability of A given B (also known as the likelihood probability) divided by the probability of A (also known as the marginal probability or normalizing constant). Each vertex (random variable) in a Bayesian network has an associated conditional probability table carrying the probability values or parameters for each value the vertex can take on.

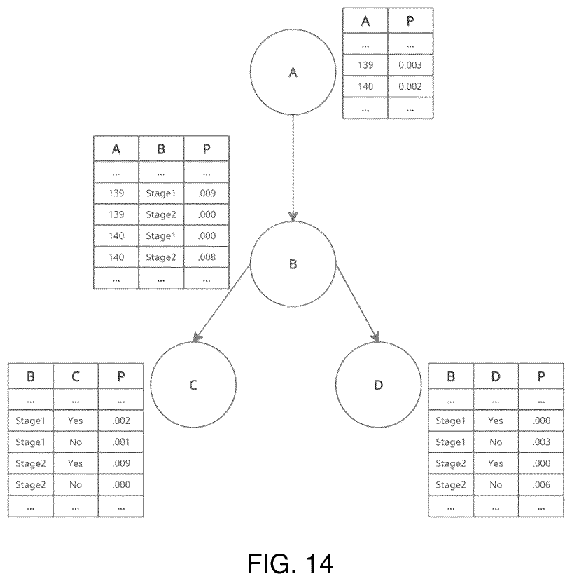

A directed acyclic graph is a set of vertices and edges. For instance, the example depicted in FIG. 14 consists of a set of vertices: [A, B, C, and D] as well as a set of edges: [A-B, B-C, B-D]. The domain of a vertex (or random variable) is the set of all the possible values the vertex can have at a given point in time. A directed acyclic graph helps simplify the computation of a joint probability distribution. FIG. 13 can be interpreted as a joint probability of specific events of each vertex occurring together at the same point in time. For instance, in a single pass throughout the network, the joint probability could be P(A=120 millimeters of mercury, B=stage 1 hypertension, C=Yes—ICD-10 documents hypertension presence 1314, D=No—medications 1316), where P stands for probability. Hence, P is a joint probability for the directed acyclic graph.

For a given P corresponding to a specific directed acyclic graph, a query consists of finding the probability of a random variable taking on a specific value from its domain (and within the joint distribution specified by P). This random variable can also be referred to as a query variable.

### Markov Condition Impact on Exponential Combinations

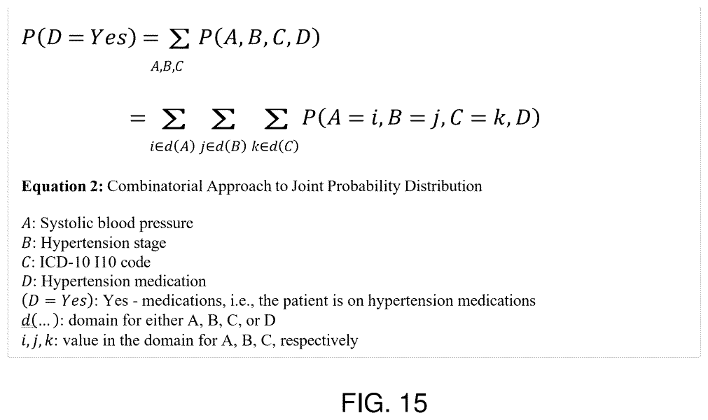

Prior to the development of Bayesian networks, to compute a query involving the conjunction of random variables required to sum over the domain of each random variable in the conjunction even when the variable is not part of the query. For instance, for the joint probability distribution P(A, B, C, D), the probability that the patient is on medication (D=Yes—medications) would be a summation over the domain of A, B, and C as shown in Equation 2 in FIG. 15. This results in a lengthy computation taking exponential-time. Nonetheless, the computation of the joint probability becomes simplified with Bayesian networks because they satisfy the Markov condition.

There should not be any cycle in a directed acyclic graph representing a Bayesian network, as per the name. Still, most importantly, the directed acyclic graph has to satisfy the Markov condition. The Markov condition states that any given vertex for any directed acyclic graph and its joint probability distribution should be independent of its non-descendant vertices given its parent vertices.

Hence, thanks to the Bayes' rule defined in Equation 3 in FIG. 16, the probability that a patient is on medication (D=Yes—medications) becomes conditioned only on the evidence (or instantiated value) of the direct parent of medication (hypertension stage or B). Formally, a conditional probability distribution is a probability distribution over the domains of specified random variables given fixed values (or instantiations) of other random variables. Indeed, Equation 3 shows a simplified derivation of the Bayes' rule whereby from the joint probability P (A, B, C, D), we can isolate the probability of hypertension stage and instantiate B as stage 1, times the joint probability of all the remaining random variables (A, C, D) given the evidence (B=stage 1). Likewise, the joint P(A, B, C, D) is also equal to the isolation of the probability that the patient is on medication (D=Yes—medications), times the joint probability of the remaining random variables (A, B, C) given the evidence (D=Yes—medications). The application of the Markov condition to the directed acyclic graph in FIGS. 13 and 14 reveals that D is independent of A and C given its parent B. Therefore, we can directly calculate the conditional probability of D given B by abstracting out the vertices that are not related to D. It follows that we can then compute the conditional probability for (D=Yes—medication) given (B=stage 1) as illustrated with the Bayes' rule in Equation 3.

The extension of the Markov condition leads to the separation of a set of vertices from another set of vertices given a third set of vertices, also called d-separation. It also allows for the Markov blanket or a set of vertices that shields a given vertex from the influence of unrelated vertices. The vertices in the shield consist of the vertex's parent, children, and children's parents.

### Marginalization and Chain Rule

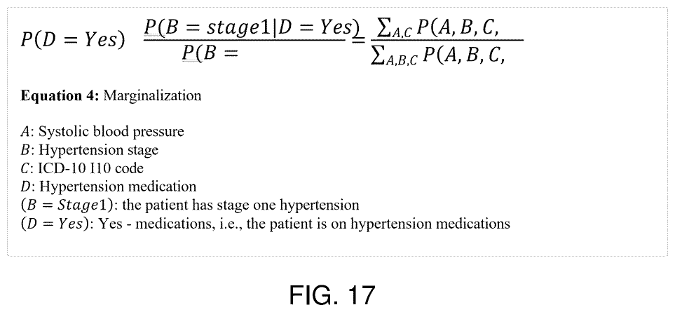

We still need to marginalize over the vertices not involved in the query, as shown in Equation 5 in FIG. 17. Marginalization means leaving out the instantiated variable from the summations. Also, inferencing means computing the value of a query, i.e., finding the probability that a given random variable has a specified value from its domain. The complexity of the combinatorial approach in Equation 2 is reduced to polynomial-time thanks to Bayesian network properties. Still, the complexity hinges on the algorithm used to conduct inferencing and the number of parent vertices that the random variable involved in the query has.

For that reason, the methods devised thus far to conduct inferencing on Bayesian networks are polynomial in time. For example, considering the directed acyclic graph in FIGS. 13 and 14, any given inferencing algorithm would compute the posterior probability of D given B within polynomial time. A problem represented via a Bayesian network is nondeterministic polynomial-time or NP-complete. In other words, even when found via approximate inferencing, the posterior probability can have its correctness verified, i.e., the exact posterior can be found in polynomial time. And one can still use a combinatorial approach (brute-force search) to find an exact posterior probability if the problem is not time-sensitive. In general, a nondeterministic polynomial-time (NP) problem contrasts with a deterministic polynomial-time problem. For the latter, all computation steps are tractable (run in polynomial-time), and each step is wholly determined by the previous step, while for the former, the problem is likely intractable and nondeterministic but can be reduced to complete in polynomial time through approximation techniques. Hence, every NP-complete problem is not just nondeterministic polynomial-time but also hard.

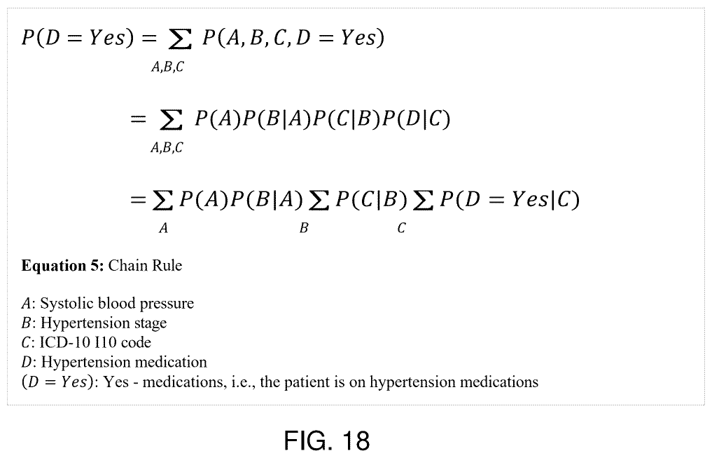

The chain rule illustrated in Equation 5 in FIG. 18 provides the basis for using marginalization to run inferences on a Bayesian network. It allows calculation of the probability of any random variable in a joint probability distribution using only conditional probabilities as expressed by Bayes' rule. The chain rule is thus a formulation of any given joint probability distribution as an incremental product of conditional probability distributions. The distributive law of elementary algebra allows distributing the different product terms in the chain rule over the summations with respect to the random variables taking part in the summations.

Inference in a Bayesian network can consist of either belief updating or revision. Belief updating is the standard approach of computing the posterior probability for some vertex in a directed acyclic graph given some evidence or known values of a set of instantiated vertices. The set could be a singleton set. Hence, belief updating is also known as probabilistic inference as it finds the posterior marginal probability of one or more random variables given the knowledge of the values of other random variables in the network. Belief revision is an inferencing technique that allows us to find the most probable value of some query variables given some evidence. It can also be called the most probable explanation or maximum a posteriori, as it can be used to find the most probable explanation or a complete assignment of the query variables that justifies the observed evidence.

There are three main classes of inference algorithms in Bayesian networks: exact inferencing, approximate inferencing, and real-time inferencing algorithms. The exact inferencing algorithms include Pearl's message passing, clique-tree propagation or clustering algorithm, arc reversal or node reduction, variable elimination, symbolic probabilistic inference, differential method, etc. Pearl's message passing and clique-tree propagation operate in a similar fashion by transforming the directed acyclic graph into some simplified graph, i.e., a polytree or single-connected graph and a clique tree, respectively. Node reduction and variable elimination are also a bit similar. They both reduce the size of the directed acyclic graph to maintain only vertices involved in the query of interest, i.e., query nodes and their parents. While variable elimination proceeds by computing marginal probabilities iteratively until the network is reduced, node reduction reverses the edges then proceeds like the variable elimination until the network is downsized. Symbolic probabilistic inference attempts to find the most optimal factoring of the chain rule, and it is thus a combinatorial optimization algorithm. After compiling the directed acyclic graph into a multivariate polynomial, the differential approach treats the query as a differentiable function by computing partial derivatives of the ensuing polynomial with respect to each variable. The posterior probability can thus be found in constant time once the derivatives have been calculated.

This technology may use a variation of the clique-tree propagation or clustering algorithm known as lazy propagation. Lazy propagation is thus an exact inferencing algorithm, and for all such algorithms, the complexity is exponential in time because they treat the query as either an NP-hard or an NP-complete problem in a bid to find an exact posterior distribution. However, it also means that lazy propagation converges slowly, but is appropriate for an application intended for a high-stakes environment, like a clinical setting.

Conversely, approximate inferencing algorithms can speed up inferencing by implementing a random search, a simulation, or a sampling technique, thus simplifying the query to at least an NP-complete problem. Approximate algorithms include stochastic simulation, model simplification, loopy propagation, etc. Simplification methods reduce the complexity of exact inferencing by annihilating small probabilities, removing weak edges based on corresponding conditional probability distributions, reducing the cardinality of conditional probability tables, etc. Stochastic simulation algorithms are also known as Monte Carlo algorithms and can further be subdivided into importance sampling and Markov chain Monte Carlo methods. Importance sampling methods encompass several variations of their own. Still, their most differentiating aspect is that the sampling is done so that samples are independent of each other. Contrariwise, Markov chain Monte Carlo methods function with dependent samples and comprise variants such as Gibbs sampling, Metropolis sampling, etc. Furthermore, whenever a Bayesian network can only be represented with a directed cyclic graph instead of an acyclic one, loopy propagation is an approximation algorithm that adapts Pearl's message propagation algorithm to solve such a network containing loops. Bayesian networks with loops can work best when modeling computer vision problems or when modeling a network to deal with error correction over noisy communication channels (or error correction codes).

The main drawback of approximate inferencing algorithms is the inability to assess whether the approximated query distribution is close to the exact distribution. However, approximate inferencing algorithms can also be used as real-time inferencing algorithms. These classes of algorithms are composed of anytime and multiple methods algorithms. The approximate inferencing algorithms are anytime algorithms because they can be run iteratively to approximate a solution, with the advantage of being stopped anytime whenever a solution needs to be used. There are also several variations of anytime algorithms, such as a combination of genetic algorithms and stochastic simulation using Monte Carlo sampling to select the most accurate probability distribution from a set of simulated probability, state-space abstraction to approximate a probabilistic query through incremental changes to the state spaces of random variables in the network, etc.

The Bayesian network inferencing techniques are not useful for learning a probability distribution or parameter learning, nor are they useful for structure learning or meta-learning to derive an optimal configuration of the directed acyclic graph. In machine learning, in general, inferencing is done after a model has been trained (or has learned) from a data set. It can thus help validate the trained model on a data set separate from the one used during training known as the development set. This is especially true when running cross-validation, which means making successive inferences during training to nudge the model to the optimal direction by trying out variation hyperparameter values. Inferencing is also needed to make predictions on new unseen data, either on a test set or in a production setting.

Nevertheless, a different set of training algorithms can be employed to train a Bayesian network, including chi-squared test. And just as for deep learning, optimization is reached thanks to an objective function. The most common objective function for training a Bayesian network to learn a joint probability distribution or parameters thereof is the maximum likelihood estimation function. The maximum likelihood estimation function maximizes the log-likelihood of the entire training data. A Bayesian network can also learn from the data it generates through random instantiations of conditional probability tables, and as such, it is known as a generative model. An alternative to the maximum likelihood estimation is the conditional log-likelihood estimation, which maximizes the conditional likelihood of a class given the input features in a classification task. Indeed, some researchers reported that the conditional likelihood estimation might perform better in classification tasks than the maximum likelihood estimation.

While the conditional likelihood estimation is good at discriminating between classes, the dynamic Bayesian belief network in this technology was trained using the maximum likelihood estimation because the model's performance improves proportionally to an increase in the training set size, and it efficiently estimates parameters in closed form as opposed to the conditional log-likelihood which cannot function in closed form due to the difficulty of decomposing each term of the logarithm of a conditional probability into individual terms corresponding to each random variable.

The maximum likelihood estimation is a frequentist method (i.e., based on relative frequencies) that approximates a random variable's probability distribution based on the occurrence count of the variable's instances in the training data. Another alternative to the maximum likelihood estimation is maximum a posteriori that is also used for inferencing to derive the most probable explanation. For parameter learning, maximum a posteriori works better for smaller data sets, whereby it is impossible to derive sufficient counts of different instances of attributes in the data. In such cases, maximum a posteriori allows us to use information about a prior joint probability distribution to estimate a new joint probability distribution.

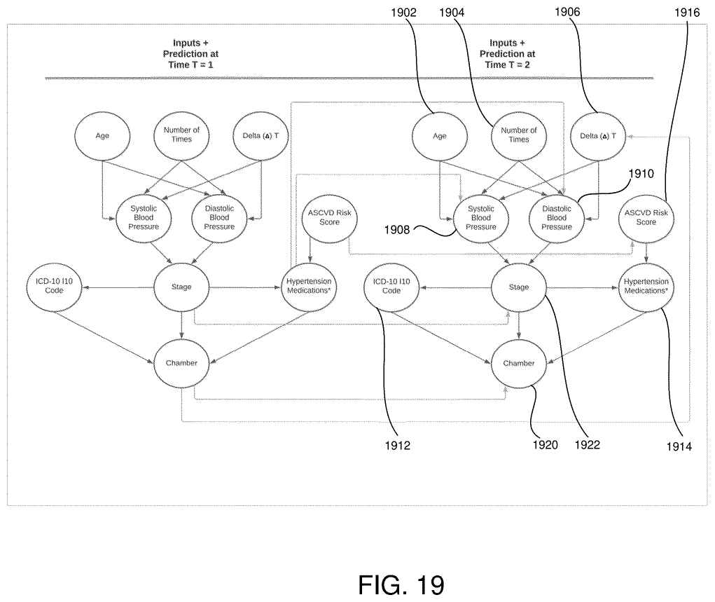

The Bayesian network structure may be defined by a structure or directed acyclic graph that is expected to perform well in a clinic, as represented in FIG. 19. However, there are methods to learn the structure of a Bayesian network. The hill-climbing algorithm enables learning the Bayesian network structure by starting from an initial structure that could be random, empty, or constructed from experts' inputs. At each iteration, a variation of the initial structure is created through the deletion or addition of a vertex or by tweaking an edge. Each new variation is assigned a score, and the training progresses until the score stops improving. The highest scoring structure is thus the optimal structure. There are several other structure learning algorithms, including one that uses the conditional log-likelihood for structure learning.

### Dynamic Bayesian Belief Network

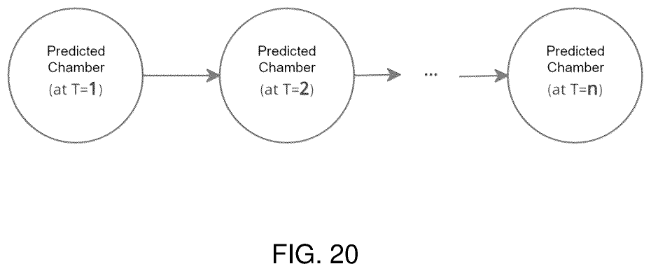

In the simplest form, a Bayesian network may consist of one random variable representing the probability distribution over the values in its domain space. For instance, FIG. 20 depicts vertices connected through a number T of time steps. This example assumes that each vertex represents the patient's chamber at a specific time step. Hence, each vertex is a standalone Bayesian network at a specific time, and the interconnected vertices are thus a representation of the simplest dynamic Bayesian network possible. Consequently, there is an assumed time series data wherein the patient's chambers are ordered from an earlier state to a later one. The network edges may represent temporal relationships between the vertices or chamber states. It follows that such a temporal connection from an earlier vertex to a later vertex implies a causal relationship between the represented random variables.

A standard (or stationary) Bayesian network implies only a dependency relationship between vertices. However, whenever parent vertices represent events that occur earlier than their corresponding child vertices, the network is thus a causal Bayesian network. For example, the Bayesian network in FIG. 19 is far more complex than the causal graph in FIG. 20 in that at any given time step there is a standard Bayesian network with ten vertices as opposed to just one node. The edges at a time step represent conditional dependencies between the vertices, as discussed above (per the depiction in FIG. 20), the vertices that connect across time steps imply that the states of the vertices at time step T=n−1 explain the states of the vertices at time step T=n.

Although they may explain the next state, edges crossing time boundaries do not necessarily imply causation. The edge between two vertices bound by time that does not model causation could be considered spurious or odd, primarily when some other vertex mediates causation. Indeed, in the causal graph in FIG. 20, considering that each vertex represents the chamber if the previous value is chamber one, that might explain why the next value is chamber two, but we cannot assert with certainty that being in chamber one at time T=n−1 caused the patient to be in chamber two at time T=n because some mediating factors such as blood pressure measurement, hypertension stage, etc. need to be considered. Hence, although the causal aspect of dynamic Bayesian networks may exist, this technology does not assume that dependency relationships (or edges) across time steps are causal.

FIG. 19 illustrates that a patient's age 1902, a number of times measurements were taken from a patient 1904 during an encounter (e.g., a time step), the time step for the current set of measurements 1906 (or time delta), a systolic blood pressure 1908, a diastolic blood pressure 1910, an ICD (International Classification of Diseases) code 1912, whether hypertension medication is being taken 1914, and an ASCVD risk score 1914 may be inputs into the Bayesian network for each time interval. In addition, the hypertension classification from a previous time step may be used in the next time step for the Bayesian network in order to obtain the final hypertension classification. Specifically, the chamber classification 1920 may use the previous chamber classification as an input. Similarly, the stage classification 1922 may use the stage from the previous time step. The previous use of hypertension medication may also be used in the probability calculations related to the systolic blood pressure 1908 and diastolic blood pressure 1910.

### Learning Parameters in Dynamic Bayesian Networks

Training a Bayesian network uses some prior knowledge about the problem at hand. This might involve finding prior knowledge about the probability distribution of the random variables involved for smaller data sets. But, regardless of the data set size, prior knowledge about the relationships between random variables is used. Although the structure can be improved through learning, starting with a structure manually constructed by domain experts leads to better results than random structure generation or starting with a blank structure.

Whenever the problem being modeled with a dynamic Bayesian network allows assuming causation, the dynamic Bayesian network can be treated as a hidden Markov model. Hence, each vertex assumes a hidden state in addition to the actual vertex instantiated value, and the sequence of these hidden states constitutes a Markov process. A Markov process describes the fact that the probability of a future event is entirely determined by the probability of the immediately preceding state.

The assumption of an acausal dynamic Bayesian network allowed experimentation with the learning algorithms. Specifically, better results may be achieved using the maximum likelihood estimation objective function. Since maximum likelihood estimation is based on counts, an implicit assumption is that a dynamic Bayesian network has to be unrolled through time before training.

However, owing to the underlying objective function, acausal dynamic Bayesian networks can learn parameters in record time compared to their deep learning counterparts. Indeed, on an equally-sized time series data set, training a dynamic Bayesian network may take a fifth of the time consumed to train a recurrent neural network.

When training a Bayesian network, it is also useful to employ a smoothing technique to avoid overfitting or fitting the parameters perfectly to the training set while generalizing poorly to the development and the test sets. Indeed, since the maximum likelihood estimation is based on relative frequencies, counting, and normalizing based on seen data (training set), it carries a great chance of overfitting. Smoothing techniques allow counteracting overfitting by assigning a small constant count to each possible value in the domains of every random variable in the network. This allows learning parameters even for unseen values during training and thus increases the chance of generalizing better to unseen data that will probably have different value counts. There exist several smoothing techniques such as Laplace smoothing, Dirichlet smoothing, a priori smoothing, etc. In our case, a priori smoothing attained better results.

Several inferencing techniques in Bayesian networks are discussed in this disclosure. Once a model has been trained, it must be run on unseen data to assess how it may perform in real life and to inform whether the training was adequate or whether more training is required. For example, each training task may be followed by an inferencing task on a separate development set to inform whether additional iterations are required to help the model improve on how the model is learning the parameters. More specifically, a variant of the clique-tree algorithm called lazy propagation may be used to conduct inferences.

The lazy propagation algorithm exploits independence properties between vertices without a direct edge to reduce the dynamic Bayesian network into a clique tree using d-separation, Markov blanket, along with several other efficiency techniques. Lazy propagation can trim down the dynamic Bayesian network in FIG. 19 in a series of successive iterations. Each iteration may retain only evidence vertices with corresponding query vertices to find new evidence or belief. The belief is propagated at subsequent iterations to unvisited vertices ignoring the visited vertices that do not constitute either the query or the evidence at the current iteration. The iterations stop when each vertex has been visited, and its probability distribution has been computed.

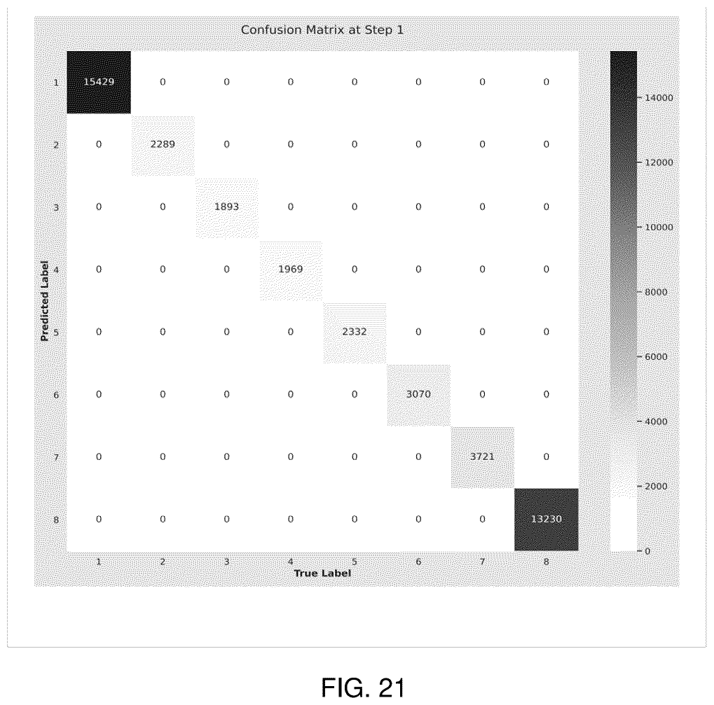

As depicted by the confusion matrix in FIG. 21, both the two-step dynamic Bayesian network and recurrent neural network models achieved perfect accuracy on the test set for this learning task. The confusion matrix is the reflection of the performance at a single time step.

In this example, a total of 12 models were trained: four dynamic Bayesian network models for each of the spinoffs in FIG. 10, eight recurrent neural network models, four for each of the variants in FIGS. 10 and 11. However, the difference in performance did not vary significantly, so only the performance on the test set from the two-step spinoff was reported.

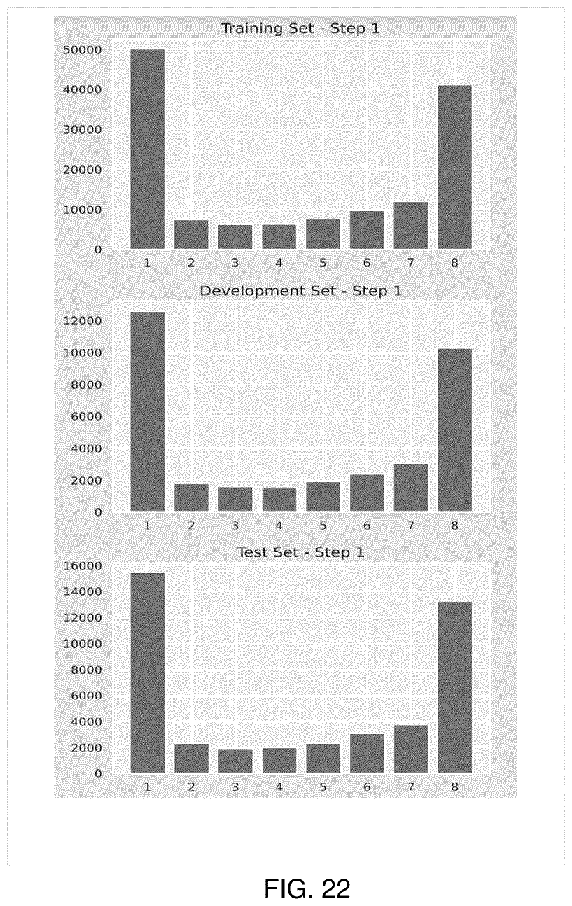

Moreover, FIG. 22 depicts the distribution of labels for the two-step spinoff. Although imbalanced, we can see a similar distribution for the training, development, and test sets at each time step. Simple random sampling was employed to select patients randomly because each patient-specific time series is unrelated to the other. A rule of thumb to follow when dealing with time series is to avoid random sampling because the time steps are correlated through time. However, in this case, the patients are not related through time, but their specific hospital visits are. Hence, it was possible to apply random sampling at the middle level (i.e., patients) in the hierarchy depicted in FIG. 6.

Training the dynamic Bayesian network may take a fraction of the time compared to the recurrent neural network. Nonetheless, the recurrent neural network may make faster inferences on a new batch of unseen data by a factor of ten or more. As described earlier, the Bayesian network learns by optimizing the maximum likelihood estimation, which essentially computes relative frequencies (of occurrences) of different values in the domains of random variables taking part in the Bayesian network. Meanwhile, the recurrent neural network minimizes the loss function, an objective function applied to individual instances in the training sample. It is the same as the cost function when applied to a batch of training instances. The specific loss function in this example is the sparse categorical cross-entropy, comparing the distribution of the model's output against a hypothetically perfect distribution of the provided labels. The model iteratively approximates the perfect distribution using gradient descent to update its parameters until the model reaches convergence. A Bayesian network may make a single pass in the data with maximum likelihood estimation to learn the relative frequencies. However, the recurrent neural network needs to see the data a few times based on the specified number of epochs.

However, the time when the recurrent neural network no longer needs to iterate several times through the entire data set is when it makes inferences. Because it has already learned the parameters, it applies them once to find the optimal output and returns the predictions in seconds or minutes depending on the data set size. On the other hand, the Bayesian network, for our use case, employs lazy propagation, an algorithm that must compute all the different conditional probability tables for the different values of random variables in the set on which the inference is being run. As discussed, most inferencing algorithms on the Bayesian network are NP-complete, especially when running an exact inference, as is the case for lazy propagation. In fact, it is during inference that a Bayesian network model will go through different iterations in the network. At each iteration, it will change the network configuration to retain only the query, and the evidence variables derive the conditional probabilities for the query then propagate them to another unseen vertex where it repeats the same process until all the vertices have been visited. The configuration change is enabled by the Markov condition discussed previously, which, in turn, enables d-separation and application of the Markov blanket.

On the upside, owing to the conditional dependencies modeled by the dynamic Bayesian networks, when the two approaches perform almost equally, the Bayesian network approach might be preferred because it natively can provide inferences that allow interpreting the output. Moreover, dynamic Bayesian networks get us a step closer to representing causal relationships, an issue of importance in medicine to better model diseases and therapies.

Deep learning approaches can also be made interpretable and explainable with added layers of computations through Shapley additive values, a concept from game theory that can assess the contributions of features to a given prediction.

The performance on the test set indicates that both machine learning models can be deployed into a real-world clinical setting and provide accurate results (given the handful of features) as long as the underlying population from which the training sample was drawn remains reasonably varied, i.e., does not vary so much as to become markedly different from its variability when the model was trained. Otherwise, the machine learning model may face a common issue in machine learning called concept drift. Concept drift happens when an AI or any statistical model displays a drop in performance proportional to the elapsing time since its deployment. With time, things change, and so do populations. The models built could also be shipped to a different region to serve a different population. In both the case for concept drift and that of applying a trained model to a different population, the model can be trained on the new data at a regular interval to maintain its performance.

The models' ability to use physician's documented hypertension (ICD-10 I10 code) as a feature to derive an even better prediction and recommendation thereof is a significant move toward collaborative AI. Collaborative AI supports a synergetic interaction between the end-user and the AI. The latter learns via human supervision to improve the cognitive ability and decision-making of the former.

Moreover, the model can output a prediction accompanied by a modifier. The modifiers are factual statements regarding a particular prediction. They may prompt the end-user to be more exhaustive when pondering on a clinical finding to devise a plan, or in this case, when diagnosing or treating hypertension. The predictions of these models are also more granular than looking at the hypertension stage alone. Indeed, hypertension status has a broad spectrum to be considered thoroughly to address the issues with misdiagnosis and under management, as we reported. The spectrum was represented as chambers and AI models to predict them accurately. The more granular the spectrum, the more nuanced the decision to predict hypertension becomes, and as such, the more important it becomes to build AI models sensible enough to capture the nuances as they may quickly become elusive to our unaided human minds.

This technology demonstrates the usage of multiple machine learning techniques to help physicians and APPs in diagnosing hypertension in an outpatient clinic. As already discussed, it is built around the end-user. It thus constitutes a step toward AI that achieves tasks alongside humans, which is foreseen to have better results in society because humans themselves have limited computational abilities. AI itself can run havoc and fail to converge, or it may converge but demonstrate critical biases in production.

However, we are limited to providing modifiers for predicted hypertension status without providing a conclusive prediction for edge cases involving masked or whitecoat hypertension. Also, the likelihood of hypertension accompanying each prediction is derived from a small number of experts for it to be irrefutably (or one hundred percent) correct.

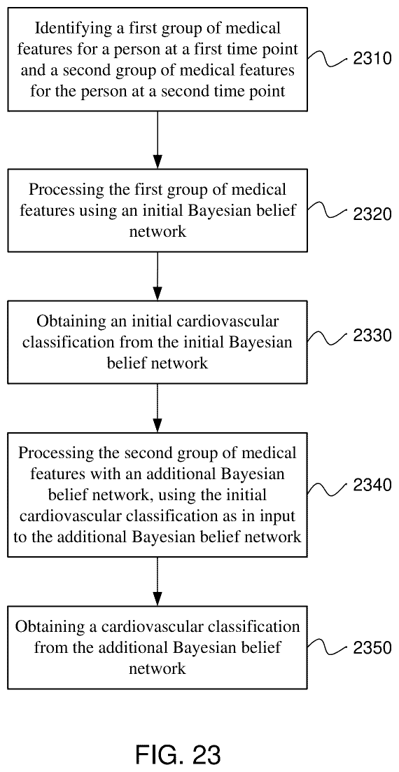

FIG. 23 is a flow chart illustrating an example of a method for identifying a cardiovascular condition. The method may include the first operation of identifying a first group of medical features for a person at a first time point and a second group of medical features for the person at a second time point, as in block 2310. The first group of medical features and the second group of medical features may include at least one of: a person's age, a number of times measurements are taken per encounter, the time difference interval, a systolic blood pressure, or a diastolic blood pressure. In one example, the first and second group of medical features may be the same medical features obtained at different points in time from a patient. Other medical features may also be used that are related to a person's cardiovascular health.

The first group of medical features may be processed using an initial Bayesian belief network, as in block 2320. The processing may result in obtaining or receiving an initial cardiovascular classification from the initial Bayesian belief network, as in block 2330. One example of a cardiovascular classification is a hypertension classification. The hypertension classification may be different states or chambers of hypertension. Additionally, hypertension may also be used to predict other types of heart disease such as coronary heart disease (CHD), heart failure, atrial fibrillation, aortic valvular disease, sudden cardiac death (SCD), sick sinus syndrome (SSS), left ventricular hypertrophy, or abdominal aortic aneurysms. Additional medical features may also be used to further diagnose heart disease or to identify cardiovascular classifications related to hypertension.

Another operation may be processing the second group of medical features with an additional Bayesian belief network, using the initial cardiovascular classification as an input to the additional Bayesian belief network, as in block 2340. In one example, the initial cardiovascular classification of the initial Bayesian belief network may be used as an input to an additional cardiovascular classification of the additional Bayesian belief network using a joint probability function. In another example, a chamber classification of the initial Bayesian belief network is an input to an additional chamber classification of the additional Bayesian belief network using a joint probability function (see FIG. 19). The chamber classification may be created using the cardiovascular classification and a treatment(s) currently being undertaken for the person.

This processing in the additional Bayesian belief network may result in obtaining a cardiovascular classification from the additional Bayesian belief network, as in block 2350. The cardiovascular classification may be a hypertension classification, a hypertension chamber, a cardiovascular classification, a cardiovascular chamber and related cardiovascular classifications.

The doctoral dissertation paper entitled “Building an AI Framework for Hypertension Diagnosis: A Use Case of the Problem List Curation” by Ketemwabi Yves Shamavu is incorporated by reference in its entirety herein.

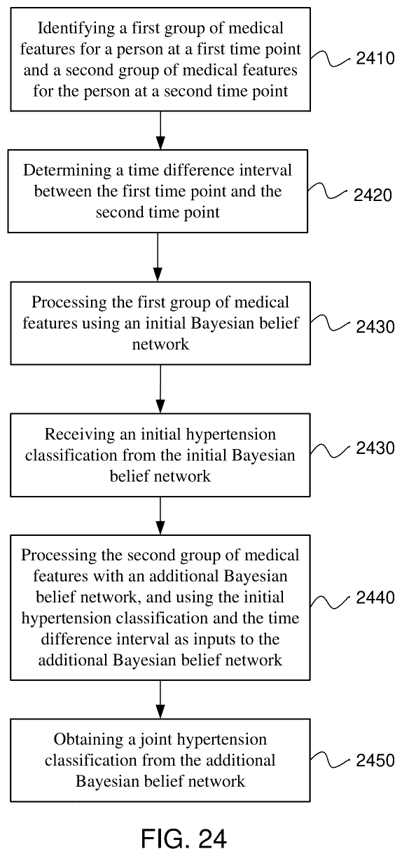

FIG. 24 is a flowchart illustrating an example of a method for identifying hypertension. The method may include identifying a first group of medical features for a person at a first time point and a second group of medical features for the person at a second time point, as in block 2410. As described earlier, the first group of medical features and the second group of medical features may include at least one of: a person's age, a number of measurements taken per encounter, the time difference interval, a systolic blood pressure, or a diastolic blood pressure.

A time difference interval between the first time point and the second time point may also be determined, as in block 2420. This may be a time difference measured in hours, days, weeks, months and/or years. The first group of medical features can be processed using an initial Bayesian belief network, as in block 2430.

Another operation may be receiving an initial hypertension classification from the initial Bayesian belief network, as in block 2440. The second group of medical features may be processed with an additional Bayesian belief network, using the initial hypertension classification and the time difference interval as inputs to the additional Bayesian belief network, as in block 2450. This can mean the initial hypertension classification of the initial Bayesian belief network is an input to an additional hypertension classification of the additional Bayesian belief network using a joint probability function. As an example, a chamber classification of the initial Bayesian belief network can be an input to an additional chamber classification of the additional Bayesian belief network using a joint probability function.

A joint hypertension classification may be obtained from the additional Bayesian belief network, as in block 2450. The joint hypertension classification is used with a therapy type to generate a chamber that is at least one of: normotensive, controlled hypertension with non-pharmacologic, controlled hypertension, medications prescribed for non-hypertensive, hypertensive but undocumented hypertension, uncontrolled hypertension, untreated hypertension, or undiagnosed/untreated hypertension.

The initial Bayesian belief network and the additional Bayesian belief network may be causal Bayesian belief networks. Parent vertices from the initial Bayesian belief network that connect to child vertices in the additional Bayesian belief network represent events that occur earlier than corresponding child vertices. A smoothing technique may also be used while training Bayesian belief networks to avoid overfitting.

This technology may provide a knowledge engine for cardiovascular disease detection that can be constructed to support a causal inference framework. The knowledge engine may support well defined clinical concepts that are understandable to clinicians and implementable by computer scientists. The knowledge base can prime the causal and Bayesian inference engines.

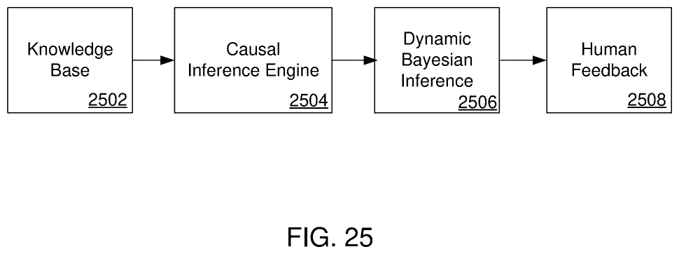

FIG. 25 is a flowchart illustrating a configuration of the present technology for a causal inference process. One operation is creating a knowledge base, as in block 2502. Published guidelines can be deconstructed into clinical concepts for the knowledge base. This knowledge base then informs the construction of the causal inference engine, as in block 2504. The causal inference engine may be trained, tested and then validated by clinical experts. The use of a dynamic Bayesian inference engine 2506 can provide the ability to model non-Gaussian continuous variables that support temporal (time and date) data and changes in conditions (new medications and therapy). The output of the causal engine is transparent in making identification of a disease classification. One goal of this technology is to automate and inform but not to replace the clinician. This may be done by having a human in the loop 2508 and obtaining human feedback or having human clinicians analyze the inputs, outputs and processes of the system.

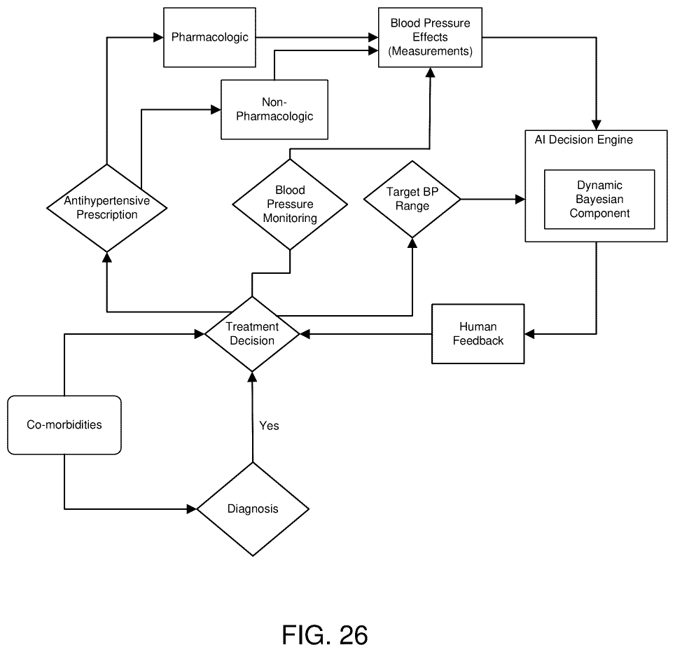

FIG. 26 is a block diagram illustrating an example of data flow and decisions for the hypertension use cases. For hypertension, the 8-chamber framework can help inform the clinically relevant diagnosis of hypertension. Thus, prescription of antihypertensive medication will be informed by co-morbid conditions (specific guideline-based, modifying conditions and follow-up testing). The measurement of blood pressures can inform the AI decision engine and makes recommendations of both dosage adjustments and frequency for blood pressure monitoring.

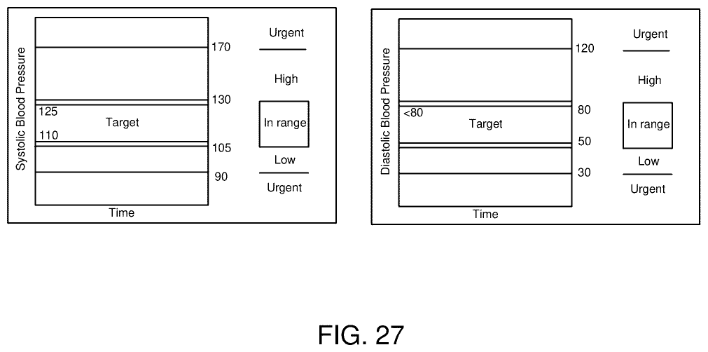

FIG. 27 illustrates that blood pressure recordings are dynamic. They are based on the individual's situation and clinical condition. For this example, use-case the blood pressure recordings have been transformed from a dichotomous decision into treatment ranges that are set by the clinician individualized to the patient.

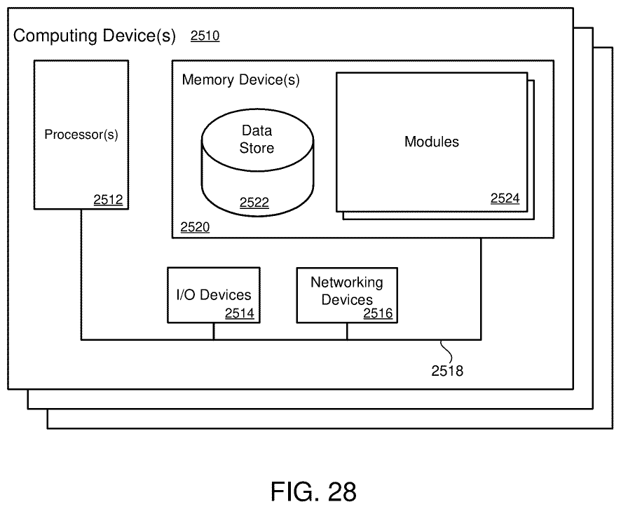

FIG. 28 illustrates a computing device 2810 on which modules of this technology may execute. The computing device 2810 is illustrated on which a high level example of the technology may be executed. The computing device 2810 may include one or more processors 2812 that are in communication with memory devices 2820. The computing device may include a local communication interface 2818 for the components in the computing device. For example, the local communication interface may be a local data bus and/or any related address or control busses as may be desired.

The memory device 2820 may contain modules 2824 that are executable by the processor(s) 2812 and data for the modules 2824. The modules 2824 may execute the functions described earlier. A data store 2822 may also be located in the memory device 2820 for storing data related to the modules 2824 and other applications along with an operating system that is executable by the processor(s) 2812.

Other applications may also be stored in the memory device 2820 and may be executable by the processor(s) 2812. Components or modules discussed in this description that may be implemented in the form of software using high programming level languages that are compiled, interpreted or executed using a hybrid of the methods.

The computing device may also have access to I/O (input/output) devices 1014 that are usable by the computing devices. An example of an I/O device is a display screen that is available to display output from the computing devices. Other known I/O device may be used with the computing device as desired. Networking devices 2816 and similar communication devices may be included in the computing device. The networking devices 2816 may be wired or wireless networking devices that connect to the internet, a LAN, WAN, or other computing network.

The components or modules that are shown as being stored in the memory device 2820 may be executed by the processor 2812. The term “executable” may mean a program file that is in a form that may be executed by a processor 2812. For example, a program in a higher level language may be compiled into machine code in a format that may be loaded into a random access portion of the memory device 2820 and executed by the processor 2812, or source code may be loaded by another executable program and interpreted to generate instructions in a random access portion of the memory to be executed by a processor. The executable program may be stored in any portion or component of the memory device 2820. For example, the memory device 2820 may be random access memory (RAM), read only memory (ROM), flash memory, a solid state drive, memory card, a hard drive, optical disk, floppy disk, magnetic tape, or any other memory components.

The processor 2812 may represent multiple processors and the memory 2820 may represent multiple memory units that operate in parallel to the processing circuits. This may provide parallel processing channels for the processes and data in the system. The local interface 2818 may be used as a network to facilitate communication between any of the multiple processors and multiple memories. The local interface 2818 may use additional systems designed for coordinating communication such as load balancing, bulk data transfer, and similar systems.

Some of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of executable code may, for instance, comprise one or more blocks of computer instructions, which may be organized as an object, procedure, or function. Nevertheless, the executables of an identified module need not be physically located together, but may comprise disparate instructions stored in different locations which comprise the module and achieve the stated purpose for the module when joined logically together.

Indeed, a module of executable code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices. The modules may be passive or active, including agents operable to perform desired functions.

The technology described here can also be stored on a computer readable storage medium that includes volatile and non-volatile, removable and non-removable media implemented with any technology for the storage of information such as computer readable instructions, data structures, program modules, or other data. Computer readable storage media include, but is not limited to, RAM, ROM, EEPROM, flash memory or other memory technology, CD-ROM, digital versatile disks (DVD) or other optical storage, magnetic cassettes, magnetic tapes, magnetic disk storage or other magnetic storage devices, or any other computer storage medium which can be used to store the desired information and described technology.

The devices described herein may also contain communication connections or networking apparatus and networking connections that allow the devices to communicate with other devices. Communication connections are an example of communication media. Communication media typically embodies computer readable instructions, data structures, program modules and other data in a modulated data signal such as a carrier wave or other transport mechanism and includes any information delivery media. A “modulated data signal” means a signal that has one or more of its characteristics set or changed in such a manner as to encode information in the signal. By way of example, and not limitation, communication media includes wired media such as a wired network or direct-wired connection, and wireless media such as acoustic, radio frequency, infrared, and other wireless media. The term computer readable media as used herein includes communication media.

Furthermore, the described features, structures, or characteristics may be combined in any suitable manner in one or more examples. In the preceding description, numerous specific details were provided, such as examples of various configurations to provide a thorough understanding of examples of the described technology. One skilled in the relevant art will recognize, however, that the technology can be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements can be devised without departing from the spirit and scope of the described technology.

What is claimed is:

1. A method for identifying a cardiovascular condition, comprising:
identifying a first group of medical features for a person at a first time point and a second group of medical features for the person at a second time point;
processing the first group of medical features using an initial Bayesian belief network;
obtaining an initial cardiovascular classification from the initial Bayesian belief network;
processing the second group of medical features with an additional Bayesian belief network, and using the initial cardiovascular classification as an input to the additional Bayesian belief network; and
obtaining a cardiovascular classification from the additional Bayesian belief network.

2. The method as in claim 1, wherein the initial cardiovascular classification of the initial Bayesian belief network is an input to an additional cardiovascular classification of the additional Bayesian belief network using a joint probability function.

3. The method as in claim 1, wherein a chamber classification of the initial Bayesian belief network is an input to an additional chamber classification of the additional Bayesian belief network using a joint probability function.

4. The method as in claim 3, wherein the chamber classification is at least one of: normotensive, controlled hypertension with non-pharmacologic, controlled hypertension, medications prescribed for non-hypertensive, hypertensive but undocumented hypertension, uncontrolled hypertension, untreated hypertension, or undiagnosed/untreated hypertension.

5. The method as in claim 1, wherein the first group of medical features and the second group of medical features include at least one of: a person's age, a number of measurements taken per encounter, a time difference interval, a systolic blood pressure, or a diastolic blood pressure.

6. The method as in claim 1, wherein the initial Bayesian belief network and the additional Bayesian belief network are causal Bayesian belief networks.

7. The method as in claim 1, wherein parent vertices from the initial Bayesian belief network that connect to child vertices in the additional Bayesian belief network represent events that occur earlier than corresponding child vertices.

8. The method as in claim 1, further comprising applying a smoothing technique while training Bayesian belief networks to avoid overfitting.

9. A method for identifying hypertension, comprising:
identifying a first group of medical features for a person at a first time point and a second group of medical features for the person at a second time point;
determining a time difference interval between the first time point and the second time point;
processing the first group of medical features using an initial Bayesian belief network;
receiving an initial hypertension classification from the initial Bayesian belief network;
processing the second group of medical features with an additional Bayesian belief network, and using the initial hypertension classification and the time difference interval as inputs to the additional Bayesian belief network; and
obtaining a joint hypertension classification from the additional Bayesian belief network.

10. The method as in claim 9, wherein the initial hypertension classification of the initial Bayesian belief network is an input to an additional hypertension classification of the additional Bayesian belief network using a joint probability function.

11. The method as in claim 9, wherein a chamber classification of the initial Bayesian belief network is an input to an additional chamber classification of the additional Bayesian belief network using a joint probability function.

12. The method as in claim 9, wherein the first group of medical features and the second group of medical features include at least one of: a person's age, a number of measurements taken per encounter, the time difference interval, a systolic blood pressure, or a diastolic blood pressure.

13. The method as in claim 9, wherein the joint hypertension classification is used with a therapy type to generate a chamber classification that is at least one of: normotensive, controlled hypertension with non-pharmacologic, controlled hypertension, medications prescribed for non-hypertensive, hypertensive but undocumented hypertension, uncontrolled hypertension, untreated hypertension, or undiagnosed/untreated hypertension.

14. The method as in claim 9, wherein the initial Bayesian belief network and the additional Bayesian belief network are causal Bayesian belief networks.

15. The method as in claim 14, wherein parent vertices from the initial Bayesian belief network that connect to child vertices in the additional Bayesian belief network represent events that occur earlier than corresponding child vertices.

16. The method as in claim 5, further comprising applying a smoothing technique while training Bayesian belief networks to avoid overfitting.

17. A machine readable storage medium having instructions embodied thereon, the instructions when executed by one or more processors, being configured for identifying a cardiovascular condition and causing the one or more processors to perform a process, comprising:
identifying a first group of medical features for a person at a first time point and a second group of medical features for the person at a second time point;
processing the first group of medical features using an initial Bayesian belief network;
obtaining an initial cardiovascular classification from the initial Bayesian belief network;
processing the second group of medical features with an additional Bayesian belief network, and using the initial cardiovascular classification as an input to the additional Bayesian belief network; and
obtaining a cardiovascular classification from the additional Bayesian belief network.

18. The machine readable storage medium as in claim 17, wherein the initial cardiovascular classification of the initial Bayesian belief network is an input to an additional cardiovascular classification of the additional Bayesian belief network using a joint probability function.

19. The machine readable storage medium as in claim 17, wherein a chamber classification of the initial Bayesian belief network is an input to an additional chamber classification of the additional Bayesian belief network using a joint probability function.

20. The machine readable storage medium as in claim 17, wherein the first group of medical features and the second group of medical features include at least one of: a person's age, a number of measurements taken per encounter, a time difference interval, a systolic blood pressure, or a diastolic blood pressure.
